SEQUENCE LISTING (11 sequences)

SEQ ID NO: 1 - DNA, 2623 bp, Homo sapiens
gaggaggcccgagaggagtcggtggcagcggcggcggcgggaccggcagcagcagcagcagcagcagcagcaaccactagcctcctgccccgcggcgttgcgacgagccccacgagccgctcaccccgccgttctcagcgctgcccgaccccgctggcgcgcctcccgccgcagtcccggcagcgcctcagttgtcctccgactcgccctcggccttcgcgcagcgcagcacagccgcacgcaccgcagcacagcacagcacagcccaggcatagcttcggcacagccccggctccggctcctgcggcagctcctctggcacgtccctgcgccgacattctggaggttggatgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctggcccgcagcttcttctggcaggtgctggaggccgtgcggcactgccacaactgcggggtgctacaccgcgacatcaaggacgaaaacatccttatcgacctcaatcgcggcgagctcaagctcatcgacttcgggtcgggggcgctgctcaaggacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatagcagcctttctggcaggtcctcccctctcttgtcagatgcccgagggaggggaagcttctgtctccagcttcccgagtaccagtgacacgtctcgccaagcaggacagtgcttgatacaggaacaacatttacaactcattccagatcccaggcccctggaggctgcctcccaacagtggggaagagtgactctccaggggtcctaggcctcaactcctcccatagatactctcttcttctcataggtgtccagcattgctggactctgaaatatcccgggggtggggggtgggggtgggcagaaccctgccaatggaactctttcttcatcatgagttctgctgaatgccgcgatgggtcaggtaggggggaaacaggttgggatgggataggactagcacattttaagtccctgtcacctcttccgactctttctgagtgccttctgtggggactccggctgtgctgggagaaatacttgaacttgcctcttttacctgctgcttctccaaaaatctgcctgggttttgttccctatttttctctcctgtcctccctcaccccctccttcatatgaaaggtgccatggaagaggctacagggccaaacgctgagccacctgcccttttttctgcctcctttagtaaaactccgagtgaactggtcttcctttttggtttttacttaactgtttcaaagccaagacctcacacacacaaaaaaatgcacaaaccaagcaatcaacagaaaagctgtaaatgtgtgtacagttggcatggtagtatacaaaaagattgtagtggatctaatttttaagaaattttgcctttaagttattttacctgtttttgtttcttgttttgaaagatgcgcattctaacctggaggtcaatgttatgtatttatttatttatttatttggttcccttcctattccaagcttccatagctgctgccctagttttctttcctcctttcctcctctgacttggggaccttttgggggagggctgcgacgcttgctctgtttgtggggtgacgggactcaggcgggacagtgctgcagctccctggcttctgtggggcccctcacctacttacccaggtgggtcccggctctgtgggtgatgggaggggccattgctgactgtgtatataggataattatgaaacacagttctggatggtgtgccttccagatcctctctggggctgtgttttgagcagcaggtagcctgctggttttatctgagtgaaatactgtacaggggaataaaagagatcttattttttttttatacttgcgtttggaataaaaaccctttggcttt

SEQ ID NO: 2 - protein, 313 aa, Homo sapiens
MLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK

SEQ ID NO: 3 - DNA, 1302 bp, Rattus norvegicus
gggatgctcttgtccaagatcaactccctggcccacctgcgcgcagccccttgcaacgacctgcacgccaacaagctggcgccgggcaaagagaaggagcccctggagtcgcagtaccaggtgggcccgctgttgggcagcggtggcttcggctcggtctactcgggcatccgcgtcgccgacaacttgccggtggccatcaagcacgtggagaaggaccggatttccgactggggggaactgcccaacggcacccgagtgcccatggaagtggtcctgctgaagaaggtgagctcgggcttctcgggcgtcattagacttctggactggttcgagaggcccgatagtttcgtgctgatcctggagaggcccgaacccgtgcaagacctcttcgacttcatcaccgagcgaggagccctccaggaggagctggcccggagcttcttctggcaggtgctggaggccgtgcggcattgccacaactgcggggttctccaccgcgacatcaaggacgagaacatcttaatcgacctgaaccgcggcgaactcaaactcatcgacttcgggtcgggggcgctgctcaaggacacagtctacacggactttgacggaacccgagtgtacagtcctccagagtggattcgctaccatcgctaccacggcaggtcggctgctgtttggtccctggggatcctgctctatgacatggtctgcggagatattccatttgagcacgacgaagagatcgtcaagggccaagtgtactttaggcaaagggtctcttcagaatgtcaacatcttattagatggtgcctgtccctgagaccatcggaccggccctcctttgaagaaatccagaaccatccgtggatgcaggatgttctcctgccccaggccaccgccgagattcatctgcacagcctgtcaccatcacccagcaaatagcagccattctgtcagaccctccagggaagagagagcttgtctgctggcctccaacaggaccctgctctacgatgcagggacagaaatgacaactcattccaggctccggggtccctggagcaacctccctcaaggagaagagactagttcactcgtcctggaccccgctttgcccctcacagactcagtggcgtccagtgtggctggcgtccgcagagtcccgggtgttggggggggaggtgggagtgggtcagagccctgtcatggaactttagtcaccatggagactgtgggtcaccaagatgggccagggtaggggaaaaacatttggggggtgggattaaaaactagcaccat

SEQ ID NO: 4 - protein, 313 aa, Rattus norvegicus
MLLSKINSLAHLRAAPCNDLHANKLAPGKEKEPLESQYQVGPLLGSGGFGSVYSGIRVADNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIVKGQVYFRQRVSSECQHLIRWCLSLRPSDRPSFEEIQNHPWMQDVLLPQATAEIHLHSLSPSPSK

SEQ ID NO: 5 - DNA, 942 bp, Mus musculus
atgctcctgtccaagatcaactccctggcccacctgcgcgcccgcccctgcaacgacctgcacgccaccaagctggcgccgggcaaagagaaggagcccctggagtcgcagtaccaggtgggcccgctgttgggcagcggtggcttcggctcggtctactctggcatccgcgtcgccgacaacttgccggtggccattaagcacgtggagaaggaccggatttccgattggggagaactgcccaatggcacccgagtgcccatggaagtggtcctgttgaagaaggtgagctcggacttctcgggcgtcattagacttctggactggttcgagaggcccgatagtttcgtgctgatcctggagaggcccgaaccggtgcaagacctcttcgactttatcaccgaacgaggagccctacaggaggacctggcccgaggattcttctggcaggtgctggaggccgtgcggcattgccacaactgcggggttctccaccgcgacatcaaggacgagaacatcttaatcgacctgagccgcggcgaaatcaaactcatcgacttcgggtcgggggcgctgctcaaggacacagtctacacggactttgatgggacccgagtgtacagtcctccagagtggattcgctaccatcgctaccacggcaggtcggcagctgtctggtcccttgggatcctgctctatgacatggtctgcggagatattccgtttgagcacgatgaagagatcatcaagggccaagtgttcttcaggcaaactgtctcttcagagtgtcagcaccttattaaatggtgcctgtccctgagaccgtcagatcggccctcctttgaagaaatccggaaccatccgtggatgcagggtgacctcctgccccaggcagcttctgagatccatctgcacagtctgtcaccgggatccagcaagtag

SEQ ID NO: 6 - protein, 313 aa, Mus musculus
MLLSKINSLAHLRARPCNDLHATKLAPGKEKEPLESQYQVGPLLGSGGFGSVYSGIRVADNLPVAIKHVEKDRISDWGELPNGTRVPMEVVLLKKVSSDFSGVIRLLDWFERPDSFVLILERPEPVQDLFDFITERGALQEDLARGFFWQVLEAVRHCHNCGVLHRDIKDENILIDLSRGEIKLIDFGSGALLKDTVYTDFDGTRVYSPPEWIRYHRYHGRSAAVWSLGILLYDMVCGDIPFEHDEEIIKGQVFFRQTVSSECQHLIKWCLSLRPSDRPSFEEIRNHPWMQGDLLPQAASEIHLHSLSPGSSK

SEQ ID NO: 7 - DNA, 1176 bp, Homo sapiens
cccacgcgtccgcagagtgccagcagctgatccggtggtgcctgtccctgcggccctcagagcggccgtcgctggatcagattgcggcccatccctggatgctgggggctgacgggggcgccccggagagctgtgacctgcggctgtgcaccctcgaccctgatgacgtggccagcaccacgtccagcagcgagagcttgtgaggagctgcacctgactgggagctaggggaccacctgccttggccagacctgggacgcccccagaccctgactttttcctgcgtgggccgtctcctcctgcggaagcagtgacctctgacccctggtgaccttcgctttgagtgccttttgaacgctggtcccgcgggacttggttttctcaagctctgtctgtccaaagacgctccggtcgaggtcccgcctgccctgggtggatacttgaaccccagacgcccctctgtgctgctgtgtccggaggcggccttcccatctgcctgcccacccggagctctttccgccggcgcagggtcccaagcccacctcccgccctcagtcctgcggtgtgcgtctgggcacgtcctgcacacacaatgcaagtcctggcctccgcgcccgcccgcccacgcgagccgtacccgccgccaactctgttatttatggtgtgaccccctggaggtgccctcggcccaccggggctatttattgtttaatttatttgttgaggttatttcctctgagcagtctgcctctcccaagccccaggggacagtggggaggcaggggagggggtggctgtggtccagggaccccaggccctgattcctgtgcctggcgtctgtcctggccccgcctgtcagaagatgaacatgtatagtggctaacttaaggggagtgggtgaccctgacacttccaggcactgtgcccagggtttgggttttaaattattgactttgtacagtctgcttgtgggctctgaaagctggggtggggccagagcctgagcgtttaatttattcagtacctgtgtttgtgtgaatgcggtgtgtgcaggcatcgcagatgggggttctttcagttcaaaagtgagatgtctggagatcatatttttttatacaggtatttcaattaaaatgtttttgtacataaaaaaaaaaaaaaaaaaaaaaaaaaaaa

SEQ ID NO: 8 - DNA, 2133 bp, Rattus norvegicus
cgctcggccagctgccgtctacgggcttccgcgcggccaccgggcaactgcgccgcgcggctgccccactgagcgctcggcctcggggccgtgggatccgccgcgctgtctgcggtcaggaagaccgccctcccgcgtccgtgccggacgggtcagaggcggcgccgcacgcgaggccacccgcgatgctgctgtccaagttcggctccctggcgcacctctgcgggcctggcggcgtggaccacctcccagtgaagatcctacagccagccaaggcggacaaggagagcttcgagaaggtgtaccaggtgggcgccgtgctcggcagcggcggcttcggcacggtctacgcgggcagccgcatcgccgacggactcccggtggctgtgaagcacgtggtgaaggagcgggtgaccgagtggggcagtctcggcggaatggccgtgcccctggaggtggtgctgctgcgcaaggtgggcgcggcgggcggcgcgcgcggcgtcatccgcctgctggactggttcgagcggcccgacggcttcctgctggtgctggagcgacccgagccggcacaggacctcttcgacttcatcactgaacgcggcgccctggacgagcctctggctcgtcgcttcttcgcgcaggtgctcgccgctgtgcggcactgccacaattgtggggtcgtgcaccgcgacatcaaggacgagaacctgctggtggacttgcgctcgggcgagctgaagctcatcgacttcggctcgggcgcggtgctcaaggacacggtctacactgactttgatggcacccgtgtgtacagccccccagagtggatccggtatcatcgatatcacgggcggtctgccactgtgtggtctctgggtgtactgctctacgacatggtgtgtggggacattccctttgagcaggatgaggagatcttgcgcggcaggctctttttccggaggagggtctccccagagtgccagcagcttattgagtggtgtctctccctgcggccctcagagaggccctcgctggaccaaattgctgcccatccctggatgctggggacagagggcagcgttccagagaactgtgaccttcggctctgtgccctggatactgatgacggagccagtaccacttccagcagtgagagcttgtgaggaggaggaggggcctggactccacactgggggcctgggctcagcctagccagccctctcccagaatgaacattttctgcctgggatgtctcctgcaaaagcagtgacctctgacccctggtgacctttgctctcggcaccgggcctgtttcctttgctttgagtgcctttttgaacgctgctccacagggcctgggttttcttgagctcttctgtccaaagatggctgcgggctaagcaaggtcccgcctgccctgggtggatacttgaacccgagaccctaccctgctgctccatcttgcggcagccttcctgaccaagtgtgtttgacatggagcgccctgtggtgcccacctccaaccctccagtctcctggtcttcgtctgggcatgtctgcacaagcaatgcaacgctgggccactgctgcccgcctgcctccctggcaccgcacgcaacgagcgtgccacggtctcttatttatggtgtgatcaccctggagggcgcccctgccctgctggggctatttattgtttaatttatttgctgaggttacttcctccaagcaaccaccttctccaggcccctggggtgttcaggaaagccaagggtggccgttcagtccacagacggcatcctggttcctgcacctgcagtaggtccctaaccccatgtttgtgggaggaggaatttgtacagtggctaatttaaggggagtgggagaccctgtcaccctgggcactctgcgctggggagggggtttaaattattgaccttgtacagtctgcttgctggctctgaaagctggggtgggggacagagtctcaagcccttaatttattttagcaactgtgttctgtgaccctggtgtgagtaggcatcaggggtggggttgtataagttcaaaagtgtgaaatgtctggagatcatattttttatacaggtatttcaattaaatgttttggtatat

SEQ ID NO: 9 - protein, 326 aa, Rattus norvegicus
MLLSKFGSLAHLCGPGGVDHLPVKILQPAKADKESFEKVYQVGAVLGSGGFGTVYAGSRIADGLPVAVKHVVKERVTEWGSLGGMAVPLEVVLLRKVGAAGGARGVIRLLDWFERPDGFLLVLERPEPAQDLFDFITERGALDEPLARRFFAQVLAAVRHCHNCGVVHRDIKDENLLVDLRSGELKLIDFGSGAVLKDTVYTDFDGTRVYSPPEWIRYHRYHGRSATVWSLGVLLYDMVCGDIPFEQDEEILRGRLFFRRRVSPECQQLIEWCLSLRPSERPSLDQIAAHPWMLGTEGSVPENCDLRLCALDTDDGASTTSSSESL

SEQ ID NO: 10 - DNA, 2447 bp, Mus musculus
gcagggcgggtgagagcgccgtgaaagccgcggaacgccgtgcacctccgcgactctactacggcaagctagtccggacgggtcgtcgtccccgcgcgccaccagcccttggtgaaacgacagggagcgtccggcttccccagcaccgccctgcgagactcaaaacagccacaccgcaaagcgagcctcgggcggaaggaggcggagcttcaggcggccccgcctccgcggaaggatacacatctccgtggtccaaaaccccggggcgaggcggccggggcgtgtgagctgctcggccagctgccgtctacgcgctttcgcgcggccaccgggcaactgcgccgcgcggctgccccgctgagcgctcggcctcggggccgtgggatccgccgcgctgtctgcggtcaggaagaccgccctcccgcgtccttgccggacgggtcagaggcggcaccgcacgcgaggccacccgcgatgctgctgtccaagttcggctccctggcgcacctctgcgggcctggcggcgtggaccacctcccagtgaagatcctacagccagccaaggctgacaaggagagcttcgagaaggtgtaccaggtgggcgccgtgctgggcagcggcggcttcggcacggtctacgcgggcagccgcatcgccgacggactcccggtggctgtgaagcacgtggtgaaggagcgggtgaccgagtggggcagtctcggcggagtggccgtgcccctggaggtggtgctgctgcgcaaggtgggcgcggcgggcggcgcgcgcggcgtcatccgcttgctggactggttcgagcggcccgacggcttcttgttggtgctggagcgacccgagccggcacaggacctcttcgacttcatcactgaacgaggcgccctggacgagccgctggcgcgtcgcttcttcgcgcaggtgcttgccgctgtgcggcactgccacaattgtggggtcgtgcaccgcgacatcaaggacgagaacctgctggtggacctgcgctcgggagagctgaagctcatcgacttcggctcgggcgcggtgctcaaggacacggtctacactgactttgatggcacccgtgtgtacagccccccagagtggatccgatatcaccgatatcacgggcggtctgccactgtgtggtctctgggtgtactgctctacgacatggtgtgtggggacattccctttgagcaggatgaggagatcttgcgcggcaggctctttttccggaggagggtctccccagagtgccagcagcttattgagtggtgtctctccctgaggccctcagagaggccctccctggaccaaattgctgcccacccctggatgctggggacagaggggagcgttccagagaactgtgaccttcggctttgtgccctggatactgacgacggagccagtaccacttccagcagtgagagcttgtgaggaggagaaggggcctgggctcggcctagccagcgctctcccagaattgaacactttctgcctgggatgtctgctgcaaaagcagtgacctctgacccctggtgacctttgctctcggcaccgggcctgtttcctttgctttgagtgcctttttgaacgctgctccacagggcctgggttttcttgagctcttctgtccaaagatggctgagggctaagcaaggtcctgccctgggtggatacttgaaccagagatcccgaccctgctgctccatctcaggaggcagccttcctgaccaagtgtgtttgacatggagcgccctgtggtgcccacctccaaccctccagtctcctggtgttcatctgggcatgtctgcacaagcaatgcaacgctgggccactgctgcccgtctgcctccccggcacggcacggctccgcacgcaacctaagcgtgccaccacggtctcttatttatggtgtgatcaccctggagggcgcccccgccctgctggggctatttattgtttaatttatttgctgaggttcctccaagcaaccaccttctccaggcccctggggtgttgaaagtcaaatgtggctgttgagtccacagacccccatcctaattcctgcacctggaggagttccccaacccccgtgtttgcgggaggaagcatttgtacagtggctaatttaaggggagtgggagaccctgtcaccctgagcactctgcgctggggaggggtttaaattattgaccttgtacagtctgcttgctggctctgaaagctggggttgggggacagagtctcaagcccttaatttattttagcagctgtgtttctgtgaccctggtgtgactaagcatcaggggtggggttgtataagttcaaaagtgtgaaatgtctgaagatcatattttttatacaggtatttcaattaaatgttttggtatataatggaaaaaaaaaaaaaaaaaaaaaaaaaaaaa

SEQ ID NO: 11 - protein, 326 aa, Mus musculus
MLLSKFGSLAHLCGPGGVDHLPVKILQPAKADKESFEKVYQVGAVLGSGGFGTVYAGSRIADGLPVAVKHVVKERVTEWGSLGGVAVPLEVVLLRKVGAAGGARGVIRLLDWFERPDGFLLVLERPEPAQDLFDFITERGALDEPLARRFFAQVLAAVRHCHNCGVVHRDIKDENLLVDLRSGELKLIDFGSGAVLKDTVYTDFDGTRVYSPPEWIRYHRYHGRSATVWSLGVLLYDMVCGDIPFEQDEEILRGRLFFRRRVSPECQQLIEWCLSLRPSERPSLDQIAAHPWMLGTEGSVPENCDLRLCALDTDDGASTTSSSESL

(19) United States
(12) Patent Application Publication
(10) Pub. No.: US 2004/0146942 A1
(43) Pub. Date: Jul. 29, 2004

(54) Screening method using PIM1-kinase or PIM3-kinase

(75) Inventors: Eberhard Weihe, Marburg (DE); Martin K.H. Schaefer, Marburg (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/705,757

(22) Filed: Nov. 12, 2003

Related U.S. Application Data
(63) Continuation of application No. PCT/EP02/05234, filed on May 13, 2002.

(30) Foreign Application Priority Data
May 11, 2001 (DE) 101 23 055.9

(51) Int. Cl. G01N 33/53

(52) U.S. Cl. 435/7.1

(57) ABSTRACT

Methods for detecting pain-relevant substances using PIM-1-kinase or PIM-3-kinase are provided. Methods of using such substances, as well as antibodies and antisense nucleotides against PIM-1-kinase or PIM-3-kinase, for instance, in pharmaceutical compositions, diagnostic agents, and in pain therapy are also provided. Certain nucleotide sequences, proteins, and part proteins are also provided.

### CROSS-REFERENCE TO RELATED APPLICATIONS

This application is a continuation of International Patent Application No. PCT/EP02/05234 filed May 13, 2002, designating the United States of America, and published in German as WO 02/093173, the entire disclosure of which is incorporated herein by reference. Priority is claimed based on Federal Republic of Germany Patent Application No. DE 101 23 055.9, filed May 11, 2001.

### FIELD OF THE INVENTION

The invention relates to methods for detecting pain-relevant substances using PIM1- or PIM3-kinase and the use of such substances, as well as antibodies and antisense nucleotides against PIM1- or PIM3-kinase in pharmaceutical formulations or compositions and diagnostic agents and in pain therapy.

### BACKGROUND OF THE INVENTION

Various pharmaceutical formulations are available for pain therapy, including acetylsalicylic acid, paracetamol, dipyrone, tramadol, morphine, and fentanyl. Substances such as amitriptyline and ketamine are also employed for the treatment of patients suffering from pain. Despite increasingly refined therapy plans, often no permanent improvement can be achieved for the patients. This is especially true for patients with chronic pain. With chronic pain, permanent changes to the involved nerve cells occurs, contributing to the problem.

Pain research in recent years has produced the fundamental finding that the development of chronic states of pain is based on plastic changes to the nervous system, in particular in the nociceptive neurons of the posterior root ganglia and the neurons in the region of the dorsal horns of the spinal cord (as an overview see: Coderre et al. 1993; Zimmermann & Herdegen, 1996). The neuronal plasticity is accompanied by changes in the expression of certain genes and leads to a long-lasting change in the phenotype of the neurons affected. The concept of neuronal plasticity has been applied to development, learning and regeneration processes. Recent findings from pain research show that this concept also intervenes in pathophysiological processes (Tölle, 1997).

The chronic development of pain has already been characterized relatively well at a phenomenological level in animal studies. Induction of chronic states of pain leads to the following changes:

Increased sensitivity and reduced stimulus threshold of peripheral nociceptors,

Activation of so-called silent nociceptors,

Reorganization of receptive fields, and

Increase in excitability in the spinal cord.

These plastic changes have been described both for the primary afferent fibers which occur in the ganglia and for the subsequent neurons located in the spinal cord, and are also assumed to occur supraspinally, e.g. in the thalamus. Like the mechanisms described for learning and memory processes, it is assumed that a specific gene program which comprises coordinated regulation of relevant genes proceeds in the cells involved. Expression of these genes contributes decisively to the pathophysiological manifestation of chronic pain.

The starting point of the invention was therefore the identification of pain-regulated genes which are modified in their expression under pain conditions and are therefore probably involved, via their regulation connections, in the development and processing of chronic pain.

Regulatory functions have already been detected for a number of known genes in various pain models (see Table 1). Certain genes for neurotransmitters (substance P, CGRP), receptors (substance P receptor, μ-, κ-, δ-opiate receptors, NMDA receptor) and transcription factors (cJun, JunB, cFos or Krox24) are examples thereof. The fact that the receptors mentioned are already used as molecular targets for the development of new analgesics (Dickenson, 1995) provides a clear indication that the identification of new pain-regulated genes is of great interest for the development of analgesics. Screening methods are of particular importance for such identification. The central idea is to interrupt the development or persistence of pain, particularly chronic pain, by influencing the function of those proteins which are formed to an increased or decreased extent in states of pain.

TABLE 1
Regulation of known genes/gene products in pain animal models

| Gene (product) | Reg | Tissue/cell | Model | Literature |
|---|---|---|---|---|
| (a) Neurotransmitters | | | | |
| CGRP | [symbol] | SC dorsal | UV | Gillardon F et al. (1992) |
| | | horn | irradiation of | |
| | | | the skin | Ann NY Acad Sci |
| | | | | 657: 493-96 |
| Preprotachykinin & | [symbol] | DRG | Monoarthritis | Donaldson LF et |
| CGRP-mRNA | | | | al. (1992) |
| | | | | Mol Brain Res |
| | | | | 16: 143-49 |
| Preprotachykinin- | [symbol] | SC dorsal | Formalin | Noguchi & Ruda |
| mRNA | | horn | | (1992) |
| | | | | J Neurosci |
| | | | | 12: 2563-72 |
| Prodynorphin mRNA | [symbol] | Spinal cord | Exp. arthritis | Hollt et al. (1987) |
| | | | | Neurosci Lett |
| | | | | 96: 247-52 |
| Dynorphin prot. | [symbol] | Spinal cord | Formalin | Ruda et al. (1988) |
| | | | | PNAS 85: 622-26 |
| Substance P | [symbol] | Nociceptors | Exp. arthritis | Levine JD et al. |
| | | | | (1984) |
| | | | | Science 226: 547-49 |
| (b) Neurotrophins | | | | |
| BDNF mRNA & | [symbol] | DRG: | intrathecal | Michael GC et al. |
| immune reactivity | | trkA+ cells | NGF inj. | (1997) |
| | | | | J Neurosci 17: |
| | | | | 8476-90 |
| (c) Receptors | | | | |
| μ-, κ-, δ-bonding | [symbol] | SC dorsal | Monoarthritis | Besse D et al. |
| | | horn | | (1992) |
| | | | | Eur J Pharmacol |
| | | | | 223: 123-31 |
| μ-Opiate receptor | [symbol] | DRG | Carrageenan | Ji R-R et al. (1995) |
| immune reactivity | | | ind. | J Neurosci |
| | | | inflammation | 15: 8156-66 |
| κ- & δ-opiate rec.- | [symbol] | DRG | Carrageenan | Ji R-R et al. (1995) |
| mRNA | | | ind. | J Neurosci |
| | | | inflammation | 15: 8156-66 |
| κ- & μ-opiate receptor- | [symbol] | SC dorsal | Monoarthritis | Maekawa K et al. |
| mRNA | | horn | | (1995) |
| | | | | Pain 64: 365-71 |
| CCK_{B}-rec. mRNA | [symbol] | DRG | Axotomy | Zhang X et al. |
| | | | | (1993) |
| | | | | Neuroscience |
| | | | | 57: 227-233 |
| NMDA-R1-mRNA | [symbol] | SC dorsal | CFA-induced | Kus L et al. (1995) |
| | | horn | inflammation | Neuroscience |
| | | laminae I | | 68: 159-65 |
| | | & II | | |
| (d) Enzymes | | | | |
| NADPH-diaphorase | [symbol] | SC dorsal | Ischiaticus | Fiallos-Estrada et |
| activity | | horn | transection | al. ('93) |
| | | | | Neurosci. Lett |
| | | | | 150: (169-73) |
| NADPH-diaphorase | [symbol] | Spinal cord | Formalin | Solodkin et al. |
| | | | | 1992 |
| | | | | Neurosci 51: 495-99 |
| NO synthetase mRNA | [symbol] | DRG | Axotomy | Verge VMK et al. |
| | | | | (1992) |
| | | | | PNAS 89: 11617-62 |
| NO synthetase protein | [symbol] | SC dorsal | Formalin | Herdegen et al. |
| | | horn | | (1994) |
| | | | | Mol Brain Res |
| | | | | 22: 245-58 |
| NO synthetase | [symbol] | DRG | Ischiaticus | Fiallos-Estrada et |
| immune reactivity | | | transection | al. ('93) |
| | | | | Neurosci Lett |
| | | | | 150: 169-73 |
| (e) Signal cascades | | | | |
| rap1A, rap1B, H-ras | [symbol] | Spinal cord | Formalin | Urayama O et al. |
| mRNA | | | | (1997) |
| | | | | Mol Brain Res |
| | | | | 45: 331-34 |
| PKC-binding | [symbol] | SC dorsal | CFA-induced | Tolle TR et al. (82) |
| | | horn | monoarthritis | J Neurol |
| | | | | 242(S2): 135 |
| (f) Transcription f. | | | | |
| cFOS | [symbol] | Spinal cord | Noxic | Hunt SP et al. |
| | | | stimulation | (1987) |
| | | | | Nature 328: 632-34 |
| cJun, JunB, cFOS | [symbol] | SC dorsal | Formalin | Herdegen T et al. |
| Krox24 | | horn | | (1994) |
| | | | | Mol Brain Res |
| | | | | 22: 245-48 |

SC, spinal cord; DRG, dorsal root ganglia; CFA, complete Freund's adjuvant; NGF, nerve growth factor

### SUMMARY OF THE INVENTION

A primary object of the invention is to provide a screening method for identification of substances relevant in pain, in particular pain-regulating substances.

A further object of the invention is to provide polynucleotides, vectors, proteins, antibodies, cells, transgenic non-human mammals, methods of therapy methods of diagnosis, methods of investigating the activity of a test substance, compounds, and formulations that are pharmaceutically or diagnostically active.

In one embodiment, the invention relates to a method for detecting pain-regulating substances with the following method steps:

(a) incubating a test substance with a cell and/or a preparation from a cell which has synthesized the protein PIM1-kinase or PIM3-kinase and/or a protein according to one of FIGS. 1b), 1d), 1f), 2c) or 2e) (SEQ ID NOS.: 2, 4, 6, 9 or 11) and/or a protein which is at least 90% homologous thereto and/or a protein encoded by a polynucleotide according to one of FIGS. 1a), 1c), 1e), 2a), 2b) or 2d) (SEQ ID NOS.: 1, 3, 5, 7, 8 or 10) or a polynucleotide which is at least 90% homologous thereto, and/or a protein which is coded by a nucleic acid which binds under stringent conditions to a polynucleotide according to one of FIGS. 1a), 1c), 1e), 2a), 2b) or 2d) (SEQ ID NOS.: 1, 3, 5, 7, 8 or 10) or antisense polynucleotides thereof, or a part protein of one of the abovementioned proteins which is at least 10 amino acids long, and

(b) measuring the binding of the test substance to the protein or part protein synthesized by the cell or measurement of at least one of the functional parameters modified by the binding of the test substance to the protein or part protein.

This novel screening method is based on the detection of potential pain activity of a substance by its interaction with a pain-regulated protein structure, in particular PIM1-kinase or related structures, or by a pain-relevant distribution of a protein structure in the CNS, in particular PIM3-kinase or related structures.

The term pain-regulating here relates to a potential regulating influence on the physiological pain event, in particular to an analgesic action. The term substance includes any compound suitable as an active compound for a pharmaceutical formulation, in particular low molecular weight active compounds, but also others, such as nucleic acids, fats, sugars, peptides or proteins, such as antibodies.

Incubation under suitable conditions means that the substance to be investigated can react with the cell or the corresponding preparation in an aqueous medium for a defined time before the measurement. The aqueous medium can be temperature-controlled here, for example between 4° C. and 40° C., and is preferably at room temperature or at 37° C. The incubation time can be varied between a few seconds and several hours, depending on the interaction of the substance with the part protein or protein. Incubation times of between 1 min and 60 min are preferred. The aqueous medium may comprise suitable salts and/or buffer systems, such that, for example, a pH of between 6 and 8, preferably pH 7.0-7.5, is maintained in the medium during the incubation. Suitable substances, such as coenzymes, nutrients, etc., may also be added to the medium. The suitable conditions can easily be optimized by persons skilled in the art as a function of the interaction of the substance to be investigated with the part protein or protein on the basis of experience, the literature, or a few simple preliminary experiments, in order to obtain the clearest possible measurement value in the method.

A cell which has synthesized a particular part protein or protein is a cell which has already expressed this part protein or protein endogenously or one which has been modified by genetic engineering such that it expresses this part protein or protein and accordingly contains the part protein or protein before the start of the method according to the invention. The cells can be from immortalized cell lines or can be native cells originating and isolated from tissues. In the latter case, the cell union is usually broken down. The preparation from these cells comprises, in particular, homogenates from the cells, the cytosol, a membrane fraction of the cells with membrane fragments, and a suspension of isolated cell organelles, etc.

The proteins and part proteins listed here have been identified in the context of this invention as regulated by pain or distributed in a pain-relevant manner by inducing pain in an animal and, after an appropriate period of time, comparing the expression pattern of the animal with that of a control animal without pain-inducing measures by sections in the spinal cord. The proteins PIM1-kinase and, in particular with respect to pain-relevant distribution, PIM3-kinase were found to have modified expression patterns.

The species from which these proteins originate is irrelevant for the functioning of the method, but it is preferable to use the human, mouse or rat variants. PIM1-kinase is known with respect to the coding DNA sequence and the amino acid sequence and is also described in its general function. This also applies in part to PIM3-kinase, in this case the as yet unknown coding DNA sequence and the amino acid sequence in the mouse and humans having been clarified in the context of this invention. However, neither of these kinases has hitherto been connected with pain and, in particular, pain regulation in the prior art. Since the identification of the proteins took place here via a modification of the expression or via the expression distribution in an in vivo pain model, for future pharmaceutical formulations using these proteins the screening method according to the invention derived therefrom has the considerable advantage of not only being built up on theoretical considerations but presumably of having a strong in vivo relevance. Since with this method the interaction of substances with proteins and peptides not hitherto used in the pain sector is rendered possible as a standard for detecting pain-regulating substances, pain-relevant substances which would not have emerged in the methods known hitherto in the prior art using other peptides or proteins are now possibly detected with this method. This is a considerable advantage of the new method provided in accordance with the invention.

The standard by which the method allows the detection of interesting substances is either the binding to the protein or part protein, which can be detected, e.g., by displacement of a known ligand, or the extent of the substance bound, or the modification of a functional parameter due to the interaction of the substance with the part protein or protein. This interaction can lie, in particular, in a regulation, inhibition and/or activation of receptors, ion channels and/or enzymes, and modified functional parameters can be, for example, the gene expression, the ionic medium, the pH or the membrane potential, or the modification of the enzyme activity or the concentration of the second messenger.

To explain the invention, in addition to the explanations given for terms in the general text, further definitions are given below in order to clarify how certain terms used in the claims in particular are to be understood and interpreted in the context of this invention.

Substance: This means a chemical compound. In one sense, these are compounds which can potentially display an action in the body, low molecular weight active compounds, nucleic acids, fats, sugars, peptides or proteins. Low molecular weight active compounds are of particular relevance.

Pain-regulating: In the context of the invention, pain-regulating means that the substance directly or indirectly influences the perception of pain. Substances with a natural analgesic action are of particular relevance.

Incubation: Incubation means the procedure in which a biological object for investigation, for example a cell or a protein, is introduced into and left in a temperature-controlled medium, such as in an incubating cabinet or on a water-bath. Suitable conditions for incubation means physiological conditions (e.g., 37° C., pH 7.2) or conditions under which an optimum measurement in the method is possible.

Cell: The cell is a self-regulating, open system which is in a flow equilibrium with its environment by permanent exchange of matter and has its own metabolism and ability to multiply. The cell can be cultured separately or can be part of a tissue, in particular from an organ, and can exist there individually or also in a cell union.

Preparation from a cell: This means preparations which are prepared by means of chemical, biological, mechanical or physical methods causing a change in the cell structure. For example, membrane fragments, isolated cell compartments, isolated cytosol, or homogenate obtained from tissue may be included.

Peptide: Means a compound of amino acids linked as a chain via peptide bonds. An oligopeptide consists of between 2 and 9 amino acids and a polypeptide of between 10 and 100 amino acids.

Protein: Means a compound of more than 100 amino acids linked as a chain via peptide bonds, with a defined spatial structure, in certain circumstances.

Part protein: Means a compound of more than 10 amino acids linked as a chain via peptide bonds, with a defined spatial structure in certain circumstances, but cut out or selected from a larger, defined protein. A part protein may be a peptide.

PIM1-kinase and PIM3-kinase: Each of these terms refers to a particular proto-oncogene and serine-threonine kinase.

Polynucleotide: Preferably, the polynucleotide is a high molecular weight polynucleotide of several nucleotides linked to one another via phosphoric acid-pentose esterification. The underlying nucleotide is in principle a base unit of nucleic acids which consists of a nucleic base, a pentose, and a phosphoric acid. However, the invention also contemplates modified polynucleotides, which retain the base sequence but have a modified backbone instead of phosphoric acid-pentose.

At least 90 (95, 97)% homologous: This means that, in their coding region, polynucleotides are at least 90% (95%, 97%) identical to the reference (figure, SEQ. ID NO., etc.) with respect to the base sequence, and in their primary structure, the sequence of amino acids, the peptides and proteins referred to are at least 90% (95%, 97%) identical to the reference.

Gene: The term gene describes a genome section with a defined nucleotide sequence which contains the information for synthesis of an m- or pre-m RNA or another RNA (e.g. tRNA, rRNA, snRNA etc). It consists of coding and non-coding sections.

Gene fragment: Means a nucleic acid section which comprises a part region of a gene in its base sequence.

Binding to the peptide, part protein or protein: This means interaction between a substance and a peptide, part protein or protein which leads to fixing.

Functional parameters: This means the measurable parameters of an experiment which correlate with the function of a protein (ion channel, receptor, enzyme).

Manipulated by genetic engineering: This means the manipulation of cells, tissues or organisms such that genetic material is introduced.

Expressed endogenously: This means the expression of a protein in a cell line under suitable culture conditions without this corresponding protein having been prompted to expression by manipulation by genetic engineering.

G protein: This means the internationally conventional abbreviation for a guanosine triphosphate (GTP)-binding protein which is activated as a signal protein by receptors coupled to the G protein.

Reporter gene: This is a general term for genes whose products can be detected easily with the aid of simple biochemical methods or histochemical methods, such as luciferase, alkaline phosphatase, or green fluorescent protein (GFP).

(Recombinant) DNA construct: This is a general term for any type of DNA molecules which are formed by in vitro linking of DNA molecules.

Cloning vector: This is a general term for nucleic acid molecules which serve as carriers of foreign genes or parts of these genes during cloning.

Expression vector: This is a term for specially constructed cloning vectors which, after introduction into a suitable host cell, allow transcription and translation of the foreign gene cloned into the vector.

LTR sequence: This is an abbreviation for long terminal repeat which is a general term for longer sequence regions which are located at both ends of a linear genome. These sequence regions occur in the genomes of retroviruses and at the ends of eukaryotic transposons, for example.

Poly A tail: Means the adenyl radicals (or residues) attached at the 3′ end of messenger RNA by polyadenylation (approximately 20-250).

Promoter sequence: A term for a DNA sequence region from where the transcription of a gene, i.e. the synthesis of the mRNA, is controlled.

ORI sequence: An abbreviation for origin of replication. The ORI sequence allows a DNA molecule to multiply as an autonomous unit in the cell.

Enhancer sequence: A term for relatively short, genetic elements, which in some cases occur as repetitions and which as a rule enhance the expression of some genes to a varying degree.

Transcription factor: A term for a protein which influences the transcription of a gene by binding to specific DNA sequences.

Culturing: This means keeping cells or tissue under suitable culture conditions.

Conditions which allow expression: This means culture conditions which allow expression of a protein of interest, which includes changes in temperature, change of medium, addition of inducing substances, and omission of inhibiting substances.

Incubation time: This means the duration of time during which cells or tissues are incubated, i.e. exposed to a defined temperature.

Selection pressure: This means the application of culture conditions which provide cells which have a particular gene product, the so-called selection marker, with a growth advantage.

Amphibia cell: A cell from an animal of the Amphibia class.

Bacteria cell: A cell which is or should be assigned to the superkingdom of Eubacteria or Arachaebacteria or originates from it.

Yeast cell: A cell which is or should be assigned to the order of the Endomycetales or originates from it.

Insect cell: A cell which is or should be assigned to the order of the Hexapoda or originates from it.

Native mammalian cell: A cell originating from a mammal which corresponds in its relevant features to the cell present in the organism.

Immortalized mammalian cell: A cell which has acquired, by the culture conditions applied or manipulation by genetic engineering, the property of dividing in the culture beyond the usual conventional frequency of division (approximately 100).

Labeled: This means rendered accessible to a detection reaction by appropriate modification or derivatization. For example radioactively, fluorescently, or luminescently.

Ligand: A substance which binds to a molecule present in the body or a cell, specifically a receptor.

Displacement: The complete or partial removal of a ligand from its binding site.

Bound activity: A measurement value which correlates with the amount of ligand bound to a receptor. The value may be determined biochemically or physically.

Regulation: The inhibition or activation of a process, preferably inhibition or activation which takes place as part of a regulating process.

Inhibition: The inhibition/reduction of a process especially as part of regulation.

Activation: Intensification, increase, or beginning a process especially as part of regulation.

Receptors: In the broadest sense, this means all the molecules present in the pro- or eukaryotic organism which can bind to an active compound. In the narrower sense, this means membrane-bound proteins or complexes of several proteins which direct a change in the cell after binding an active compound.

Ion channels: Membrane-bound proteins or complexes of several proteins which permit cations or anions to pass through the membrane.

Enzymes: A term for proteins or complexes of an activating non-protein component with a protein having catalytic properties.

Gene expression (express/expressible): The translation of the genetic information of a gene into RNA (RNA expression) or into protein (protein expression).

Ionic medium: An ion concentration of one or more ions in a particular compartment.

Membrane potential: A potential difference over a membrane on the basis of an excess of cations on one side and anions on the other side of the membrane.

Change in enzyme activity: The inhibition or induction of the catalytic activity of an enzyme.

2nd messenger: A small molecule which, as a response to an extracellular signal, either is formed in the cytosol or migrates into the cytosol and thereby helps to transmit information to the inside of the cell, for example, adenosine-3′,5′-cyclic monophosphate (cyclic AMP or cAMP) or inositol triphosphate (IP₃).

(Gene) probe: A term for any type of nucleic acids which enable detection of a gene or a particular DNA sequence. By derivatization of the gene probe (e.g., using biotin, magnetic beads, or digoxinin), DNA molecules can furthermore be drawn out of a mixture. Cloned genes, gene fragments, chemically synthesized oligonucleotides and also RNA, which is usually radioactively labeled, are used as probes.

DNA: An international term for deoxyribonucleic acid.

Genomic DNA: A general term for the DNA originating from the cell nucleus of a cell in eukaryotic organisms.

cDNA: An abbreviation for complementary DNA. This means the single- or double-stranded DNA copy of an RNA molecule.

cDNA bank/library: A term for a collection of arbitrarily cloned cDNA fragments which, taken together, represent the entirety of all the RNA synthesized by a cell or a tissue.

cDNA clone: A term for a population of genetically uniform cells which are derived from a single cell such that this cell contains an artificially introduced cDNA fragment.

Hybridization: The formation, effected by base pairing, of a double-stranded nucleic acid molecule from two separate single strands.

Stringent conditions: Conditions under which only perfectly base-paired nucleic acid strands are formed and remain stable.

Isolate: To separate from a mixture.

DNA sequencing: The determination of the sequence of bases in a DNA molecule.

Nucleic acid sequence: A term for the primary structure of a DNA molecule, i.e., the sequence of the individual bases from which a DNA molecule is composed.

Gene-specific oligonucleotide primer: Oligonucleic acids, preferably nucleic acid fragments 10-40 bases long, which, in their base composition, allow a stringent hybridization to the gene sought or the cDNA sought.

Determination of oligonucleotide primers: A manual or computer-assisted search of oligonucleotides for a given DNA sequence which are of optimum suitability for a hybridization and/or a polymerase chain reaction.

PCR: An abbreviation for polymerase chain reaction. The PCR is an in vitro process for selective concentration of nucleic acid regions of defined length and defined sequence, especially from a mixture of nucleic acid molecules.

DNA template: A nucleic acid molecule or a mixture of nucleic acid molecules from which a DNA section is multiplied with the aid of the PCR (see above).

RNA: An internationally common abbreviation for ribonucleic acids.

mRNA: An internationally common abbreviation for messenger ribonucleic acids which are involved in transfer of the genetic information from the nucleus into the cell and contain information for the synthesis of a polypeptide or a protein.

Antisense polynucleotide: A molecule comprising several natural or modified nucleic acids, the base sequence of which is complementary to the base sequence of a part region of an RNA which occurs in nature.

PNA: An internationally common abbreviation for peptidic nucleic acids. Peptidically linked amino acids form a chain, and the different bases are linked so that the molecule is capable of hybridization with DNA or RNA.

Sequence: A sequence of nucleotides or amino acids. In the specific context of this invention, this means the nucleic acid sequence.

Ribozyme: A term for a catalytically active ribonucleic acid (e.g., ligase, endonuclease, polymerase, or exonuclease).

DNA enzyme: A term for a DNA molecule which contains catalytic activity (e.g., ligase, endonuclease, polymerase, or exonuclease).

Catalytic RNA/DNA: A general term for ribozymes or DNA enzymes (see above).

Adenovirus: A cytopathogenic virus which occurs in vertebrates.

Adeno-associated virus (AAV): Means a virus in the family of Parvoviruses. For effective multiplication of an AAV, co-infection of the host cells with helper viruses (e.g. herpes, vaccinia or adeno-viruses) is necessary. The AAV integrates into the host genome in a stable manner making it of particular interest as a transduction vector for mammalian cells.

Herpes virus: The viral pathogen of herpes infection

Post-translational modification: A modification to proteins or polypeptides carried out after translation, which includes phosphorylation, glycosylation, amidation, acetylation, or proteolysis.

Glycosylate: To append individual sugar molecules or whole sugar chains to proteins.

Phosphorylate: To append one or more phosphate radicals to a protein, preferably to the OH groups of the amino acids serine, threonine, or tyrosine.

Amidate: To convert a carboxyl function into an amide function, e.g. on the carboxy-terminal amino acid radical of a peptide or protein.

Provided with a membrane anchor: This means a post-translational modification of a protein or of another organic molecule such that, by appending a hydrophobic molecule, such as a fatty acid or a derivative thereof, it is anchored to the lipid double-layer membrane of cells.

Cleave: To cleave a peptide or protein into several sub-sequences.

Shorten: To shorten a molecule consisting of several individual parts by one or more parts.

Antibodies: This means proteins, called immunoglobulins, which are soluble or bound to cell membranes and have a specific binding site for antigens.

Monoclonal antibodies: Antibodies which have an extremely high selectivity and are directed against a single antigenic determinant, or epitope, of an antigen.

Polyclonal antibodies: A mixture of antibodies directed against several determinants, or epitopes, of an antigen.

Transgenic: This means genetically modified.

Non-human mammal: The entirety of mammals (class of Mammalia) with the exception of the human species.

Germ cell: A cell with a haploid genome which, by fusion with a second germ cell, renders possible the formation of a new organism.

Somatic cell: A diploid cell that is a constituent of an organism.

Chromosomal introduction: An intervention in the nucleotide sequence at the chromosomal level.

Genome: A general description of the entirety of all the genes in an organism.

Ancestor of the animal: An animal (the ancestor) which is related in a direct line with another animal (the descendant) in a natural or artificial manner by passing on its genetic material.

Expressible: A nucleic acid molecule is expressible if it contains the information for synthesis of a protein or polypeptide and is provided with appropriate regulatory sequences which allow synthesis of this protein or polypeptide in vitro or in vivo. If these prerequisites no longer exist, for example by intervention into the coding sequence, the nucleic acid molecule is no longer expressible.

Rodent: An animal from the order of the Rodentia, e.g. rat or mouse.

Substance which can be identified as pain-regulating: A substance which, when introduced into a living organism, causes a change in behavior which one skilled in the art would deem pain-inhibiting (antinociceptive, antihyperalgesic, or antiallodynic). In the case of the screening method, this relates to the fact that, during screening, the substance significantly, for example by 100%, exceeds the binding or interaction of the average substances tested due to stronger binding or inducement of a modification in a functional parameter.

Compound: Another name for a molecule consisting of several atoms, preferably a molecule identified by the method according to the invention.

Active compound: A compound which, when used on an organism, causes a change in this organism. In particular, this means molecules synthesized by organic chemistry which have a healing action on the organism. Molecules which bind to the proteins and peptides according to the invention are particularly preferred.

Low molecular weight: A molecule with a molecule weight of <2 kDa.

Medicament: A substance corresponding to the definition in article 1 §2 of the Act on Circulation of Medical Preparations.

Diagnostic agent: A compound or method which can be used to diagnose a disease.

Treatment of pain: A method with the aim of alleviating or eliminating pain or inhibiting the expected occurrence of pain (pre-emptive analgesia).

Chronic pain: A pain sensation of long-lasting duration, often characterized in that it increases the pain sensitivity of the body beyond the point in time and location of the initial stimulus.

Gene therapy: Gene therapy refers to those methods which have the aim of causal treatment of genetic diseases by suitable modifications to the genome.

In vivo gene therapy: The introduction of genetic material into the living organism with the aim of gene therapy. A distinction can be made between somatic and germ path intervention, which takes place in the former instance on diploid cells and in the latter instance on haploid cells.

In vitro gene therapy: The introduction of genetic material into cells outside the human body with the aim of subsequently using these again for gene therapy by introduction into the human body.

Diagnostics: Methods for identifying a disease.

Investigation of activity: An investigation with the aim of investigating the activity of a compound after it acts on a living organism.

In a preferred embodiment of the method, the cell is manipulated by genetic engineering before step (a) above. In this procedure, genetic material is introduced into the cell, in particular one or more polynucleotide sequences. In a variant of this embodiment which is even more preferred, the manipulation by genetic engineering permits the measurement of at least one of the functional parameters modified by the test substance. In this embodiment, prerequisites under which the modification of a functional parameter can be measured at all or in an improved manner are created by manipulation by genetic engineering. It is particularly preferable here for a form of a G protein which is not expressed endogenously in the cell to be expressed or a reporter gene to be introduced by the manipulation by genetic engineering. This is to be understood, in particular, as meaning the introduction into the cell, by genetic engineering, of a G protein (guanosine 5′-triphosphate-binding protein or GTP-binding protein) which is not present endogenously or is not expressed physiologically. For example, a chimeric G protein which allows a modification of the signal path or a promiscuous G protein which binds very readily may be introduced. The introduction of a reporter gene permits measurement of an (extracellularly triggered) induced expression of the gene product.

In another embodiment of the invention, the cell is manipulated by genetic engineering such that the cell contains at least one polynucleotide according to one of FIGS. 1a), 1c), 1e), 2a), 2b) or 2d) (SEQ ID NOS. 1, 3, 5, 7, 8 or 10) or a polynucleotide which is at least 90% homologous thereto. As a result, a part protein or protein which is not expressed endogenously in the cell or preparation used in the method may be synthesized by the cell. It is particularly preferable for the polynucleotide to be contained in a recombinant DNA construct. A (recombinant) DNA construct means a DNA molecule prepared in vitro.

If the cell is manipulated by genetic engineering before step a) in the method, it is preferable for the cell to be cultured, after the manipulation by genetic engineering and before step a), under conditions which allow expression. The culture conditions may optionally provide for selection pressure as a means for keeping cells or tissue under conditions which ensure survival of the cells or their subsequent generation. The conditions for culturing are chosen to facilitate expression of the material inserted by the manipulation by genetic engineering. Preferably, the pH, oxygen content and temperature are maintained at physiological values and sufficient nutrients and necessary cofactors are provided. The selection pressure allows only the cells in which the manipulation by genetic engineering was at least partly successful to be cultured further. Selection pressure includes, for example, the use of an antibiotic resistance introduced via the DNA construct.

In a particularly preferred process according to the invention, the cell is an amphibia cell, bacterial cell, yeast cell, insect cell or an immortalized or native mammalian cell. Examples for amphibia cells are Xenopus oocytes; for bacteria cells, E. coli cells; for yeast cells, those from Saccharomyces cerevisiae; for insect, cells Sf9 cells; for immortalized mammalian cells, HeLa cells; and for native mammalian cells, the CHO (Chinese hamster ovary) cell.

In a preferred measurement method according to one embodiment of the invention, a determination of the binding of a substance to a part protein or protein is carried out. This is done by detecting the displacement of a known labeled ligand of the part protein or protein and/or by detecting the activity bound thereto with a labeled test substance. A ligand refers to a molecule which binds to the protein or part protein with a high specificity and is displaced from the binding site by a substance to be tested which also binds. Labeling means an artificial modification of the molecule which facilitates detection. Examples of labeling include radioactive, fluorescent, or luminescent labeling.

In another preferred measurement method in accordance with an embodiment of the invention, a determination of the modification of the functional parameter induced by the binding of the substance to the part protein or protein is achieved. The measurement of at least one of the functional parameters modified by the test substance is carried out by measurement of the regulation, inhibition and/or activation of receptors, ion channels and/or enzymes. More particularly, the method may include measurement of the modification in gene expression, the ionic medium, the pH or the membrane potential, of a modification in the enzyme activity or the concentration of the second messenger. This includes on one hand, measurement of the action of the substance directly upon receptors, ion channels and/or enzymes, and on the other hand, measurement of parameters such as gene expression, ionic medium, pH, membrane potential, enzyme activity or concentration of the second messenger. The latter measurements are preferred. Ionic medium here means the concentration of one or more ions in a cell compartment, in particular the cytosol. Membrane potential means the charge difference between two sides of a biomembrane. Second messenger is understood here as meaning messenger substances of the intracellular signal path, e.g., cyclic AMP (cAMP), (IP₃) or diacylglycerol (DAG).

In a preferred variant of the process, the part protein or protein in steps (a) and (b) is chosen from:

PIM1-kinase,

a protein encoded by a polynucleotide according to one of FIGS. 1a), 1c) or 1e) (SEQ ID NOS. 1, 3 or 5) or a polynucleotide which is at least 90%, preferably at least 95%, and even more preferably at least 97% homologous thereto,

a protein with an amino acid sequence according to one of FIGS. 1b), 1d) or 1f) (SEQ ID NOS. 2, 4 or 6), or a protein which is at least 90%, preferably at least 95%, and even more preferably at least 97% homologous thereto, and/or

a protein which is coded by a nucleic acid which binds under stringent conditions to a polynucleotide according to one of FIGS. 1a), 1c) or 1e) (SEQ ID NOS. 1, 3 or 5) or antisense polynucleotides thereof,

or a part protein of one of the abovementioned proteins which is at least 10 amino acids long.

This method includes the use of part proteins and, in particular, proteins with a known sequence and function which are not known for having a pain-related function in the prior art.

The invention also preferably provides a polynucleotide which is at least 90%, preferably at least 95%, and even more preferably at least 97% homologous to one of the nucleotide sequences shown in one of FIGS. 2a) and 2d) (SEQ ID NOS. 7 and 10). The gene fragments shown are themselves included, as is a polynucleotide which corresponds either completely to or at least to parts of the coding sequence of the gene corresponding to the fragment. This also means polynucleotides which are at least 90%, preferably at least 95%, and even more preferably at least 97% homologous, in the base sequence, with the coding sequence of the polynucleotides shown or the coding sequence of the gene.

The polynucleotide is preferably RNA or single- or double-stranded DNA, in particular mRNA or cDNA.

In another preferred embodiment, the polynucleotide is an antisense polynucleotide or PNA which has a sequence which is capable of binding specifically to a polynucleotide according to the invention. PNA is understood here as meaning “peptidic nucleic acid”, which carries the base pairs but has a backbone of peptidic bonds. The antisense polynucleotide has a base sequence that is complementary to at least a part of a base nucleic acid. It is also preferable for the polynucleotide to be part of a ribozyme or other DNA enzyme or of a catalytic RNA or DNA. Ribozyme means a catalytically active ribonucleic acid, and DNA enzyme means a corresponding deoxyribonucleic acid, i.e., catalytic RNA and DNA respectively.

The invention also provides a vector containing one of the polynucleotides described above. A vector means a nucleic acid molecule which, during manipulation by genetic engineering, serves to contain or transfer foreign genes. It is particularly preferable here for this to be an expression vector. The expression vector may then serve to express the foreign gene contained therein, preferably the polynucleotide.

Preferably, the vector is derived from a virus, for example adenovirus, adeno-associated virus or herpes virus, and/or contains at least one LTR, poly A, promoter and/or ORI sequence. An LTR is a “long terminal repeat”, a section at the end of the sequence, for example in viruses. A Poly A sequence is a tail more than 20 adenosine radicals (or residues) long. A promoter sequence is the control region for transcription.

In another embodiment, the invention provides a protein or a part protein derived therefrom which is coded by one of the polynucleotides previously described.

In another embodiment, the invention provides a protein or a part protein derived therefrom encoded by a polynucleotide which hybridizes under stringent conditions with one of the polynucleotides according to FIG. 2a) or 2d) (SEQ ID NOS. 7 or 10) or the antisense polynucleotide thereof.

In another embodiment, the invention provides a protein or a part protein derived therefrom which is at least 90%, preferably at least 95%, and even more preferably at least 97% homologous to one of the amino acid sequences shown in FIG. 2e) (SEQ ID NO. 11).

In another embodiment, the invention provides a protein or a part protein derived therefrom which has been modified post-translationally, including proteins or part proteins which have been glycosylated, phosphorylated, amidated, methylated, acetylated, adenosine diphosphate-ribosylated (ADP-ribosylated), hydroxylated, provided with a membrane anchor, cleaved or shortened. Post-translational modifications may be found, for example, in Voet/Voet, Biochemistry, 1st Edition, 1990, p. 935-938, the disclosure of which is incorporated herein by reference.

In another embodiment, the invention provides antibodies against a protein or part protein such as that described previously. Preferably the antibody is a monoclonal or polyclonal antibody.

In another embodiment, the invention provides a cell containing a polynucleotides, a protein or part protein and/or a vector, in each case, as described previously herein. Preferably the cell is an amphibia cell, bacteria cell, yeast cell, insect cell or an immortalized or native mammalian cell. Examples for amphibia cells are Xenopus oocytes; for bacteria cells, E. coli cells; for yeast cells, those from Saccharomyces cerevisiae; for insect cells, Sf9 cells; for immortalized mammalian cells, HeLa cells; and for native mammalian cells, the CHO (Chinese hamster ovary) cell.

Preferably, the cell contains a particularly selected form of the polynucleotide, a particularly selected form of the protein or part protein and/or a particularly selected form of the vector.

The invention also provides a transgenic non-human mammal with the germ and somatic cells having, as a result of a chromosomal introduction into the genome of the animal or the genome of one of the ancestors of the animal, one of the polynucleotides described previously. Chromosomal introduction means intervention by manipulation by genetic engineering which has an effect in the chromosome of the animal.

In another embodiment, the invention provides a transgenic non-human mammal with germ and somatic cells which, as a result of a chromosomal manipulation in the genome of the animal or in the genome of one of the ancestors of the animal, no longer contain in an expressible form one of the polynucleotides described previously. Preferably, the absent polynucleotides are not antisense nucleotides. Even more preferably, the absent polynucleotides do not have sequences according to FIG. 2a) or 2d) (SEQ ID NOS. 7 or 10). The chromosomal manipulation affects either the gene of the animal or of its ancestor. No longer expressible means that the information for synthesis of a polypeptide or protein, although present in the native form, no longer allows the complete synthesis. This may be achieved by modification of the regulatory sequences or excision of part of the native nucleic acid molecule in the coding region.

Preferably the transgenic non-human mammal is a rodent.

In another embodiment, the invention provides a compound which can be identified as a pain-regulating substance by a method according to the invention. Compound here relates in particular to low molecular weight active compounds, and also to peptides, proteins, and nucleic acids. Identifiable here means that, in the screening method according to the invention, the compound binds significantly more strongly than the substances tested. Preferably, the compound binds twice as strongly as the average of the substances being tested, or with respect to the modification of the functional parameters, it deviates significantly from the average of the substances being tested.

In one embodiment, a compound according to the invention can be identified as a pain-regulating substance by a method using a protein or part protein in steps (a) and (b) which is chosen from:

PIM1-kinase,

a protein encoded by a polynucleotide according to one of FIGS. 1a), 1c) or 1e) (SEQ ID NOS. 1, 3 or 5) or a polynucleotide which is at least 90%, preferably at least 95%, and even more preferably at least 97% homologous thereto,

a protein with an amino acid sequence according to one of FIGS. 1b), 1d) or 1f), or a protein which is at least 90%, preferably at least 95%, or even more preferably at least 97% homologous thereto, and/or

a protein which is coded by a nucleic acid which binds under stringent conditions to a polynucleotide according to one of FIGS. 1a), 1c) or 1e) (SEQ ID NOS. 1, 3 or 5) or antisense polynucleotides thereof, or

a part protein of one of the abovementioned proteins which is at least 10 amino acids long.

In another embodiment, the invention provides a pharmaceutical formulation comprising:

a. a polynucleotide which codes for PIM1-kinase or PIM3-kinase or a polynucleotide which is at least 90%, preferably at least 95%, or even more preferably at least 97% homologous to one of the nucleotide sequences shown in one of FIGS. 1a), 1c), 1e), 2a), 2b) or 2d) (SEQ ID NOS. 3, 5, 7, 8 or 10),

b. a polynucleotide, in particular an antisense polynucleotide or PNA, which has a sequence which is capable of binding specifically to one of the polynucleotides listed under point a),

c. a vector containing a polynucleotide according to one of points a) or b),

d. a PIM1-kinase or PIM3-kinase and/or a protein according to one of FIGS. 1b), 1d), 1f), 2c) or 2e) (SEQ ID NOS. 2, 4, 6, 9 or 11) and/or a protein which is at least 90% homologous to one of the abovementioned proteins and/or a protein encoded by a polynucleotide according to one of FIGS. 1a), 1c), 1e), 2a), 2b) or 2d) (SEQ ID NOS. 1, 3, 5, 7, 8 or 10) or a polynucleotide which is at least 90% homologous thereto, and/or a protein which is coded by a nucleic acid which binds under stringent conditions to a polynucleotide according to one of FIGS. 1a), 1c), 1e), 2a), 2b) or 2d) (SEQ ID NOS. 1, 3, 5, 7, 8 or 10) or antisense polynucleotides thereof or a part protein of one of the abovementioned proteins which is at least 10 amino acids long,

e. an antibody against one of the proteins or part proteins according to point d),

f. a cell containing a polynucleotide according to one of points a) or b), a vector according to point c), a protein or part protein according to point d) or an antibody according to point e),

g. a compound which has been identified as a pain-regulating substance by a method according to the invention, and/or

h. an active compound which binds to a protein or part protein according to point a),

and optionally suitable auxiliary substances and/or additives. The pharmaceutical formulations according to the invention can be administered as liquid pharmaceutical formulations in the form of injection solutions, drops or juices or as semi-solid pharmaceutical formulations forms in the form of granules, tablets, pellets, patches, capsules, plasters or aerosols. The pharmaceutical formulations optionally comprise carrier materials, fillers, solvents, diluents, dyestuffs and/or binders, depending on the pharmaceutical form. The choice of the auxiliary substances and the amounts thereof to be employed depend on whether the pharmaceutical formulations are to be administered orally, perorally, parenterally, intravenously, intraperitoneally, intradermally, intramuscularly, intranasally, buccally, rectally or locally, for example to infections on the skin, the mucous membranes and the eyes. Formulations in the form of tablets, coated tablets, capsules, granules, drops, juices and syrups are suitable for oral administration, and solutions, suspensions, easily reconstitutable dry formulations and sprays are suitable for parenteral, topical and inhalatory administration. In one embodiment, the pharmaceutical formulation is suitable for percutaneous administration and is provided in a depot in dissolved form or in a plaster, optionally with the addition of agents which promote penetration through the skin. Formulation which can be used orally or percutaneously can release their active ingredient or compound in a delayed manner. The amount of active compound to be administered to the patient varies as a function of the weight of the patient, the mode of administration, the indication and the severity of the disease. 2 to 500 mg/kg of at least one of the items of subject matter according to the invention are conventionally administered. If the pharmaceutical formulation is to be used for gene therapy in particular, a physiological saline solution, stabilizers, proteinase inhibitors, DNAse inhibitors, etc., are recommended as suitable auxiliary substances, additives, or adjuvants.

In another embodiment, the invention provides a diagnostic agent comprising at least

a. a polynucleotide which codes for PIM1-kinase or PIM3-kinase or a polynucleotide which is at least 90%, at least preferably at least 95%, and even more preferably at least 97% homologous with one of the nucleotide sequences shown in one of FIGS. 1a), 1c), 1e), 2a), 2b) or 2d) (SEQ ID NOS. 1, 3, 5, 7, 8 or 10),

b. a polynucleotide, preferably an antisense polynucleotide or PNA, which has a sequence which is capable of binding specifically to one of the polynucleotides listed under point a),

c. a vector containing a polynucleotide according to one of points a) or b),

d. a PIM1-kinase or PIM3-kinase and/or a protein according to one of FIGS. 1b), 1d), 1f), 2c) or 2e) (SEQ ID NOS. 2, 4, 6, 9 or 11) and/or a protein which is at least 90% homologous to one of the abovementioned proteins and/or a protein encoded by a polynucleotide according to one of FIGS. 1a), 1c), 1e), 2a), 2c) or 2e) (SEQ ID NOS. 1, 3, 5, 7, 8 or 10) or a polynucleotide which is at least homologous thereto 90%, and/or a protein which is coded by a nucleic acid which binds under stringent conditions to a polynucleotide according to one of FIGS. 1a), 1c), 1e), 2a), 2b) or 2d) (SEQ ID NOS. 1, 3, 5, 7, 8, or 10) or antisense polynucleotides thereof or a part protein of one of the abovementioned proteins which is at least 10 amino acids long,

e. an antibody against one of the proteins or part proteins according to point d),

f. a cell containing a polynucleotide according to one of points a) or b), a vector according to point c), a protein or part protein according to point d) or an antibody according to point e),

g. a compound which has been identified as a pain-regulating substance by a method according to the invention, and/or

h. an active compound which binds to a protein or part protein according to point a),

and optionally, suitable additives. Diagnostic agent means any aid for diagnosis, for example, diagnosis of a disease process.

A form of the diagnostic agent which comprises a polynucleotide which is an antisense polynucleotide or PNA is also provided in accordance with an embodiment of the invention.

In another embodiment, the invention provides a method of preparing a pharmaceutical formulation and a method of treating pain by administering

a. a polynucleotide which codes for PIM1-kinase or PIM3-kinase or a polynucleotide which is at least 90%, preferably at least 95%, and even more preferably at least 97% homologous to one of the nucleotide sequences shown in one of FIGS. 1a), 1c), 1e), 2a), 2b) or 2d) (SEQ ID NOS. 1, 3, 5, 7, 8 or 10),

b. a polynucleotide, preferably an antisense polynucleotide or PNA, which has a sequence which is capable of binding specifically to one of the polynucleotides listed under point a),

c. a vector containing a polynucleotide according to one of points a) or b),

d. a PIM1-kinase or PIM3-kinase and/or a protein according to one of FIGS. 1b), 1d), 1f), 2c) or 2e) (SEQ ID NOS. 2, 4, 6, 9 or 11) and/or a protein which is at least 90% homologous to one of these abovementioned proteins and/or a protein encoded by a polynucleotide according to one of FIGS. 1a), 1c), 1e), 2a), 2b) or 2d) (SEQ ID NOS. 1, 3, 5, 7, 8 or 10) or a polynucleotide which is at least 90% homologous thereto and/or a protein encoded by a nucleic acid which binds under stringent conditions to a polynucleotide according to one of FIGS. 1a), 1c), 1e), 2a), 2b) or 2d) (SEQ ID NOS. 1, 3, 5, 7, 8 or 10) or antisense polynucleotides thereof or a part protein of one of the abovementioned proteins which is at least 10 amino acids long,

e. an antibody against one of the proteins or part proteins according to point d),

f. a cell containing a polynucleotide according to one of points a) or b), a vector according to point c), a protein or part protein according to point d) or an antibody according to point e),

g. a compound which has been identified as a pain-regulating substance by a method according to the invention, and/or

h. an active compound which binds to a protein or part protein according to point a).

Pharmaceutical formulations of the present invention are useful for treatment of chronic, in particular neuropathic or inflammation-induced, pain.

In yet another embodiment, the invention provides a method for gene therapy comprising administering

a. a polynucleotide which codes for PIM1-kinase or PIM3-kinase or a polynucleotide which corresponds to the extent of at least 90%, preferably at least 95%, and even more preferably at least 97% homologous to one of the nucleotide sequences shown in one of FIGS. 1a), 1c), 1e), 2a), 2b) or 2d) (SEQ ID NOS. 1, 3, 5, 7, 8 or 10),

b. a polynucleotide, preferably an antisense polynucleotide or PNA, which has a sequence which is capable of binding specifically to one of the polynucleotides listed under point a),

c. a vector containing a polynucleotide according to one of points a) or b),

f. a cell containing a polynucleotide according to one of points a)or b) or a vector according to point c).

It is particularly preferable here for the therapy to be in vivo or in vitro gene therapy. Gene therapy means a therapy form in which an effector gene, usually a protein, is expressed by introduction of nucleic acids into cells. A distinction is made in principle between in vivo and in vitro methods. In the case of in vitro methods, cells are removed from the organism and transfected ex vivo with vectors, in order to be subsequently introduced again into the same or into another organism. In the case of in vivo gene therapy, vectors, for example for combating tumors, are administered systemically (e.g., via the bloodstream) or directly into the target tissue (e.g., into a tumor).

For gene therapy, the use of a polynucleotide which is an antisense polynucleotide or PNA, or is part of a ribozyme or other DNA enzyme or of a catalytic RNA or DNA is also preferred.

The invention also provides a method for diagnostics and/or for investigations of activity comprising administering an active ingredient which is

a. a polynucleotide which codes for PIM1-kinase or PIM3-kinase or a polynucleotide which is at least 90%, preferably at least 95%, and even more preferably at least 97% homologous to one of the nucleotide sequences shown in one of FIGS. 1a), 1c), 1e), 2a), 2b) or 2d) (SEQ ID NOS. 1, 3, 5, 7, 8 or 10),

b. a polynucleotide, preferably an antisense polynucleotide or PNA, which has a sequence which is capable of binding specifically to one of the polynucleotides listed under point a),

c. a vector containing a polynucleotide according to one of points a) or b),

d. a PIM1-kinase or PIM3-kinase and/or a protein according to one of FIGS. 1b), 1d), 1f), 2c) or 2e) (SEQ ID NOS. 2, 4, 6, 9 or 11) and/or a protein which is at least 90% to one of these abovementioned proteins and/or protein encoded by a polynucleotide according to one of FIGS. 1a), 1c), 1e), 2a), 2b) or 2d) (SEQ ID NOS. 1, 3, 5, 7, 8 or 10) or a polynucleotide which is at least 90% homologous thereto, and/or a protein which is encoded by a nucleic acid which binds under stringent conditions to a polynucleotide according to one of FIGS. 1a), 1c), 1e), 2a), 2b) or 2d) (SEQ ID NOS. 1, 3, 5, 7, 8 or 10) or antisense polynucleotides thereof or a part protein of one of the abovementioned proteins which is at least 10 (preferably 20) amino acids long,

e. an antibody against one of the proteins or part proteins according to point d),

f. a cell containing a polynucleotide according to one of points a) or b), a vector according to point c), a protein or part protein according to point d) or an antibody according to point e),

g. a compound which has been identified as a pain-regulating substance by a method according to the invention, and/or

h. an active compound which binds to a protein or part protein according to point a).

Then, measuring a change in a functional parameter caused by the active ingredient.

Diagnostics means the analysis of symptoms assigned to a disease syndrome, and investigations of activity means investigations of the activity of substances to be tested, in particular their medicinal activity.

In another embodiment, the invention provides a process for the preparation of a peptide or protein, in which a cell according to the invention, which contains a polynucleotide according to the invention and/or a vector according to the invention, is cultured and the peptide or protein is optionally isolated.

The invention also provides a method for investigating the activity of a test substance or detecting a pain-regulating substance comprising the steps of administering or incubating a test substance with an indicator which is

a. a polynucleotide which codes for PIM1-kinase or PIM3-kinase or a polynucleotide which is at least 90%, preferably at least 95%, and even more preferably at least 97% homologous to one of the nucleotide sequences shown in one of FIGS. 1a), 1c), 1e), 2a), 2b) or 2d) (SEQ ID NOS. 1, 3, 5, 7, 8 or 10),

b. a polynucleotide, preferably an antisense polynucleotide or PNA, which has a sequence which is capable of binding specifically to one of the polynucleotides listed under point a),

c. a vector containing a polynucleotide according to one of points a) or b),

d. a PIM1-kinase or PIM3-kinase and/or a protein according to one of FIGS. 1b), 1d), 1f), 2c) or 2e) (SEQ ID NOS. 2, 4, 6, 9 or 11) and/or a protein which is at least 90% homologous to one of the abovementioned proteins and/or a protein encoded by a polynucleotide according to one of FIGS. 1a), 1c), 1e), 2a), 2b) or 2d) (SEQ ID NOS. 1, 3, 5, 7, 8 or 10) or a polynucleotide which is at least 90% homologous thereto, and/or a protein which is coded by a nucleic acid which binds under stringent conditions to a polynucleotide according to one of FIGS. 1a), 1c), 1e), 2a), 2b) or 2d) (SEQ ID NO. 1, 3, 5, 7, 8 or 10) or antisense polynucleotides thereof or a part protein of one of the abovementioned proteins which is at least 10 (preferably 20) amino acids long,

e. an antibody against one of the proteins or part proteins according to point d) and/or

f. a cell containing a polynucleotide according to one of points a) or b), a vector according to point c), a protein or part protein according to point d) or an antibody according to point e).

Then, optionally, measuring the binding of the test substance with the indicator or measuring at least one functional parameter modified by the binding of the test substance to the indicator.

For a pharmaceutical formulation according to the invention, a diagnostic agent according to the invention and/or other methods according to the invention, it is particularly preferable for the polynucleotide according to point a) to be a polynucleotide which codes for PIM1-kinase or a polynucleotide which is at least 90%, preferably at least 95%, and even more preferably at least 97% homologous to one of the nucleotide sequences shown in one of FIGS. 1a), 1c) or 1e) (SEQ ID NOS. 1, 3 or 5), and/or for the protein according to point d) to be a PIM1-kinase and/or a protein according to one of FIGS. 1b), 1d) or 1f) (SEQ ID NOS. 2, 4 or 6) and/or a protein which is at least 90% homologous and/or a protein encoded by one of these abovementioned proteins homologous to a polynucleotide according to one of FIGS. 1a), 1c) or 1e) (SEQ ID NOS. 1, 3 or 5) or a polynucleotide at least 90% homologous thereto, and/or a protein which is coded by a nucleic acid which binds under stringent conditions to a polynucleotide according to one of FIGS. 1a), 1c) or 1e) (SEQ ID NOS. 1, 3 or 5) or antisense polynucleotides thereof or a part protein of one of the abovementioned proteins which is at least 10 amino acids long.

For a polynucleotide according to the invention (including antisense polynucleotides, for instance), a pharmaceutical formulation according to the invention, a diagnostic agent according to the invention and/or a use according to the invention, it is particularly preferable for the polynucleotide (optionally according to point a) and/or point b)) to be an RNA or a single- or double-stranded DNA, in particular mRNA or cDNA.

For a polynucleotide according to the invention (not including antisense polynucleotides), a pharmaceutical formulation according to the invention, a diagnostic agent according to the invention and/or a use according to the invention, it is particularly preferable for the polynucleotide (optionally according to point b)) to be part of a ribozyme or other DNA enzyme or of a catalytic RNA or DNA.

For a vector according to the invention, a pharmaceutical formulation according to the invention, a diagnostic agent according to the invention and/or a use according to the invention, it is particularly preferable here for the vector (optionally according to point c)) to be an expression vector.

For a vector according to the invention, a pharmaceutical formulation according to the invention, a diagnostic agent according to the invention and/or a use according to the invention, it is furthermore particularly preferable here for the vector (optionally according to point c)) to be derived from a virus, for example adenovirus, adeno-associated virus or herpes virus and/or to contain at least one LTR, poly A, promoter and/or ORI sequence.

For a protein or part protein according to the invention, a pharmaceutical formulation according the invention, a diagnostic agent according to the invention and/or a use according to the invention (not including gene therapy), it is particularly preferable here for the protein or part protein (optionally according to point d)) to have been modified post-translationally, in particular to have been glycosylated, phosphorylated, amidated, methylated, acetylated, ADP-ribosylated, hydroxylated, provided with a membrane anchor, cleaved or shortened.

For an antibody according to the invention (also antisense etc.), a pharmaceutical formulation according to the invention, a diagnostic agent according to the invention and/or a use according to the invention (not including gene therapy), it is particularly preferable here for the antibody (optionally according to point e)) to be a monoclonal or polyclonal antibody.

For a cell according to the invention, a pharmaceutical formulation according to the invention, a diagnostic agent according to the invention and/or a use according to the invention, it is particularly preferable here for the cell (optionally according to point f)) to be an amphibia cell, bacteria cell, yeast cell, insect cell or an immortalized or native mammalian cell.

For a compound according to the invention (also antisense etc.), a pharmaceutical formulation according to the invention, a diagnostic agent according to the invention and/or a use according to the invention, it is particularly preferable here for the compound (optionally according to point g)) to be a low molecular weight compound.

For a pharmaceutical formulation according to the invention, a diagnostic agent according to the invention and/or a use according to the invention, it is particularly preferable here for the active compound mentioned, according to point h), to be a low molecular weight active compound.

In a particularly preferred embodiment of a method according to the invention, a part method is provided and the protein or part protein in steps (a) and (b) is chosen from:

PIM1-kinase,

a protein encoded by a polynucleotide according to one of FIGS. 1a), 1c) or 1e) (SEQ ID NOS. 1, 3 or 5) or a polynucleotide which is at least 90%, preferably at least 95%, and even more preferably at least 97% homologous thereto,

a protein with an amino acid sequence according to one of FIGS. 1b), 1d) or 1f) (SEQ ID NOS. 2, 4 or 6) or a protein which is at least 90%, preferably at least 95%, and even more preferably at least 97% homologous thereto, and/or

a protein which is coded by a nucleic acid which binds under stringent conditions to a polynucleotide according to one of FIGS. 1a), 1c) or 1e) (SEQ ID NOS. 1, 3 or 5) or antisense polynucleotides thereof,

or a part protein of one of the abovementioned proteins which is at least 10 amino acids long

and/or in another part method the protein or part protein in steps (a) and (b) is chosen from:

PIM2-kinase, or

PIM3-kinase,

a protein encoded by a polynucleotide according to one of FIGS. 2a), 2b) or 2d) (SEQ ID NOS. 7, 8 or 10) or a polynucleotide which is at least 90%, preferably at least 95%, and even more preferably at least 97% homologous thereto,

a protein with an amino acid sequence according to one of FIGS. 2c) or 2e) (SEQ ID NO. 9 or 11) or a protein which is at least 90%, preferably at least 95%, and even more preferably at least 97% homologous thereto and/or

a protein which is coded by a nucleic acid which binds under stringent conditions to a polynucleotide according to one of FIGS. 2a), 2b) or 2d) (SEQ ID NOS. 7, 8 or 10) or antisense polynucleotides thereof,

or a part protein of one of the abovementioned proteins which is at least 10 amino acids long,

the results from step b) of the part methods being compared differentially—usually subsequently.

The invention also provides a process for treatment, in particular pain treatment, of a non-human mammal or human which or who requires treatment of pain, in particular chronic pain, by administration of a pharmaceutical formulation according to the invention, in particular one comprising a substance according to the invention and/or an active compound according to the invention.

The administration can take place, for example, in the form of a pharmaceutical formulation as described above.

Overall, an important concern of the invention is the identification of pain-regulated genes and gene fragments. The screening method is based on this. However, the invention is also useful for diagnosis or therapy, as already stated. Appropriate possible uses and further embodiments and examples are provided hereafter.

1. Therapy of Chronic Pain

mRNA expression of kinases was investigated by in situ hybridization in spinal cord tissue. In the spinal cord, the primary sensory neurons project to subsequent central nervous neurons, these being, in addition to supraspinal processes, the central switching site for nociceptive information. Numerous experiments have shown that the development of chronic states of pain is based on plastic changes in the nervous system (as an overview see Corderre et al., 1993; Zimmermann and Herdegen, 1996). In the neurons of the dorsal root ganglia and spinal cord in particular, plastic changes which are accompanied by regulation of pain-relevant genes have been described. Gene regulation in the spinal cord has thus been described for a number of neurotransmitter receptors which are of importance for pain therapy (see Table 1). On this basis, the cDNA sequences found which are regulated under pain could be used for therapy (gene therapy, antisense, ribozymes) and diagnosis of chronic states of pain.

1.1 Antisense Strategies

Constructs which are derived from the nucleic acid sequence of the complete cDNA or from part regions and which can reduce the mRNA or protein concentration may be useful in accordance with certain embodiments of the invention. These can be, e.g., antisense oligonucleotides (DNA or RNA), which have an increased stability towards nucleases, possibly using modified nucleotide units (e.g. O-allyl-ribose). Furthermore, the use of ribozymes, which, as enzymatically active RNA molecules, catalyze a specific cleavage of the RNA, is conceivable. In addition, vectors which express the sequences according to the invention or part regions of these nucleotide sequences under control of a suitable promoter and are therefore suitable for an in vivo or ex vivo therapy could also be employed. Antisense constructs which, under exchange of the phosphate backbone of nucleotide sequences (e.g. PNAs, i.e. peptide nucleic acid) or by using non-traditional bases, such as inosines, quenosines or wybutosines as well as acetyl-, methyl-, thio- and similarly modified forms of adenine, cytidine, guanosine, thymidine and uridine, cannot be degraded or can be degraded to a relatively low degree by endogenous nucleases are additionally also possibly useful.

1.2. Antagonists/Agonists or Inhibitors/Activators of the Gene Products According to the Invention Used in the Screening Method.

This includes substances which, by binding to the gene product, modify the function thereof. These can be:

1.2.1. Organic chemical molecules which are found in the context of an active compound screening using the gene products of the cDNA according to the invention as binding partners.

1.2.2. Antibodies, whether polyclonal, chimeric, single-chain, F_{ab} fragments (antigen binding fragments) or fragments from phage banks, which preferably specifically influence the function as neutralizing antibodies via binding to the gene products.

1.2.3. Aptamers, i.e. nucleic acids or nucleic acid derivatives with protein-binding properties. These also include so-called mirror-mers, which are mirror-image and therefore stable oligonucleotides obtained by mirror evolution and can bind a target molecule with a high affinity and high specificity (see, e.g., Kluβmann et al., 1996, the disclosure of which is incorporated herein by reference).

1.3. Gene Therapy

The sequences described can be employed for therapy of neurological diseases, in particular chronic states of pain, by using them, after cloning into suitable vectors (e.g. adenovirus vectors or adeno-associated virus vectors), for in vivo or ex vivo therapy in order e.g. to counteract an over-expression or under-expression of the endogenous gene product, to correct the sequence of the defective gene product (e.g. by trans-splicing with the exogenous construct) or provide a functional gene product.

2. Diagnosis

Polynucleotide sequences (oligonucleotides, antisense DNA and RNA molecules, PNAs) which are derived from the nucleotide sequences used in the screening method could be employed for diagnosis of states or diseases associated with an expression of these gene sequences. Examples of these states or diseases include neurological diseases, including chronic pain or neuropathic pain (caused e.g. by diabetes, cancer or AIDS), or neurodegenerative diseases, such as Alzheimer's, Parkinson's, Huntington's Chorea, Jacob-Creutzfeld, amyotrophic lateral sclerosis and dementias. The nucleotide sequences can serve in diverse ways (northern blot, southern blot, FISH analysis, PRINS analysis PCR) either for identification of the gene product or deviating diagnostically relevant gene products or for quantification of the gene product. In addition to nucleic acid diagnostics, antibodies or aptamers against the protein coded by the nucleic acids according to the invention can also be employed for diagnostics (e.g. by means of ELISA, RIA, immunocytochemical or immunohistochemical methods) in order to identify the protein or deviating forms and to quantify the protein.

With respect to gene diagnostics, nucleic acid probes derived from the nucleotide sequences according to the invention could be employed for determination of the gene locus (e.g. by FISH, FACS, artificial chromosomes, such as YACs, BACs or P1 constructs).

Certain embodiments of the present invention may be understood more readily by reference to the following figures and the specific examples. The terminology used herein is for the purpose of describing particular embodiments only and is not intended to be limited.

### BRIEF DESCRIPTION OF THE DRAWINGS

FIG. 1a) cDNA sequence of PIM1-kinase, human; AN: NM_{—}002648; SEQ ID NO. 1;

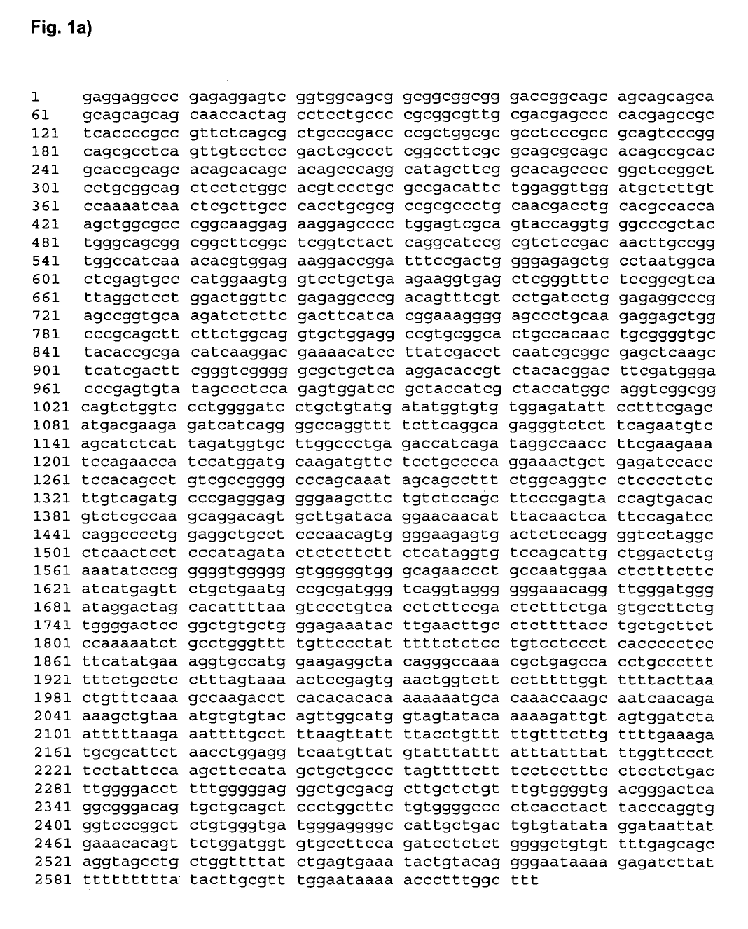

FIG. 1b) Amino acid sequence of PIM1-kinase, human; AN: NP_{—}002639; SEQ ID NO. 2;

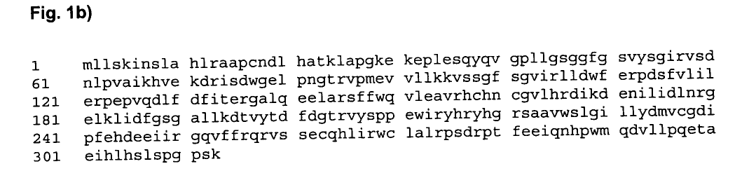

FIG. 1c) cDNA sequence of PIM1-kinase, rat; AN: NM_{—}017034; SEQ ID NO. 3;

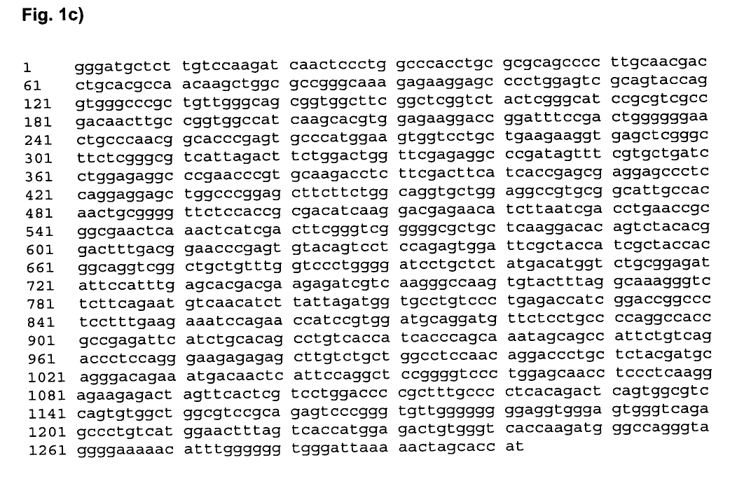

FIG. 1d) Amino acid sequence of PIM1-kinase, rat; AN: NP_{—}058730; SEQ ID NO. 4;

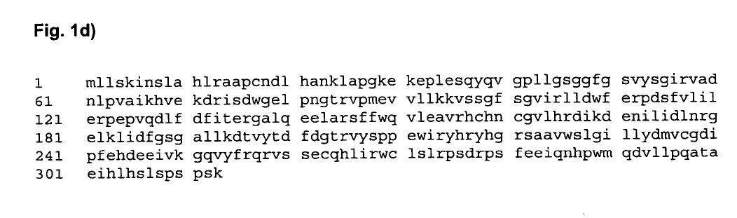

FIG. 1e) cDNA sequence of PIM1-kinase, mouse; AN: NM_{—}008842; SEQ ID NO. 5;

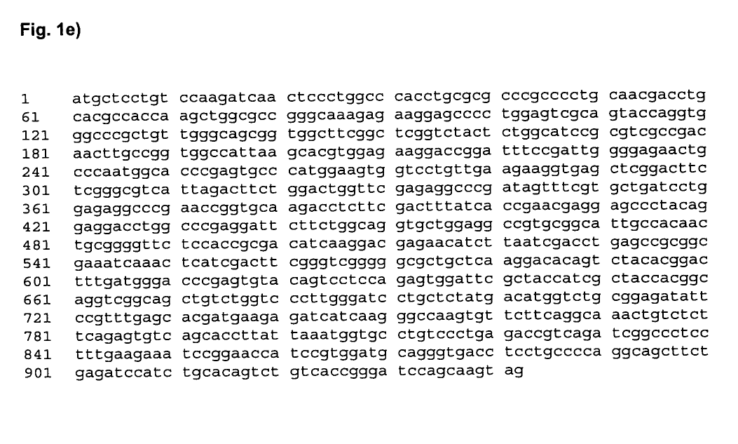

FIG. 1f) Amino acid sequence of PIM1-kinase, mouse; AN: NP_{—}032868; SEQ ID NO. 6;

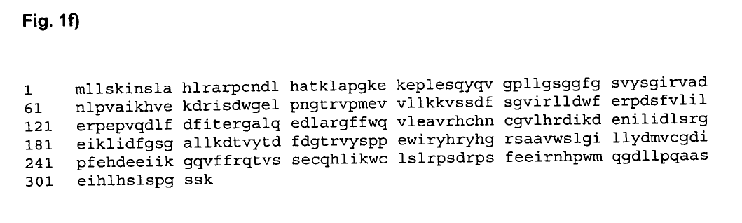

FIG. 2a) mRNA sequence similar to PIM3-kinase, human; AN: BC017083; SEQ ID NO. 7;

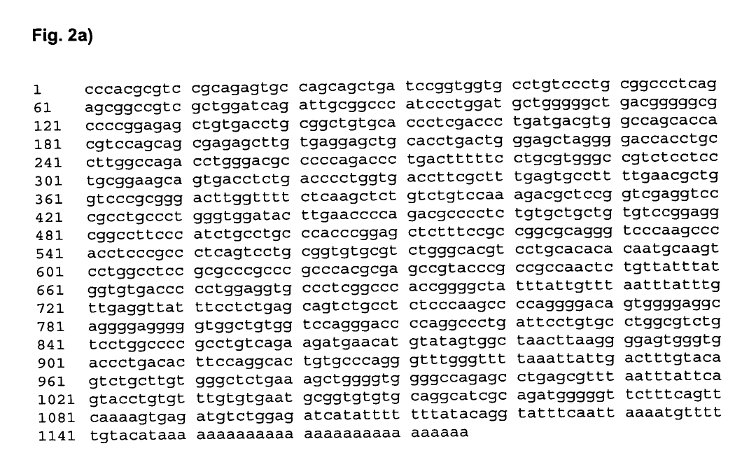

FIG. 2b) cDNA sequence of PIM3-kinase, rat; AN: NM_{—}022602; SEQ ID NO. 8

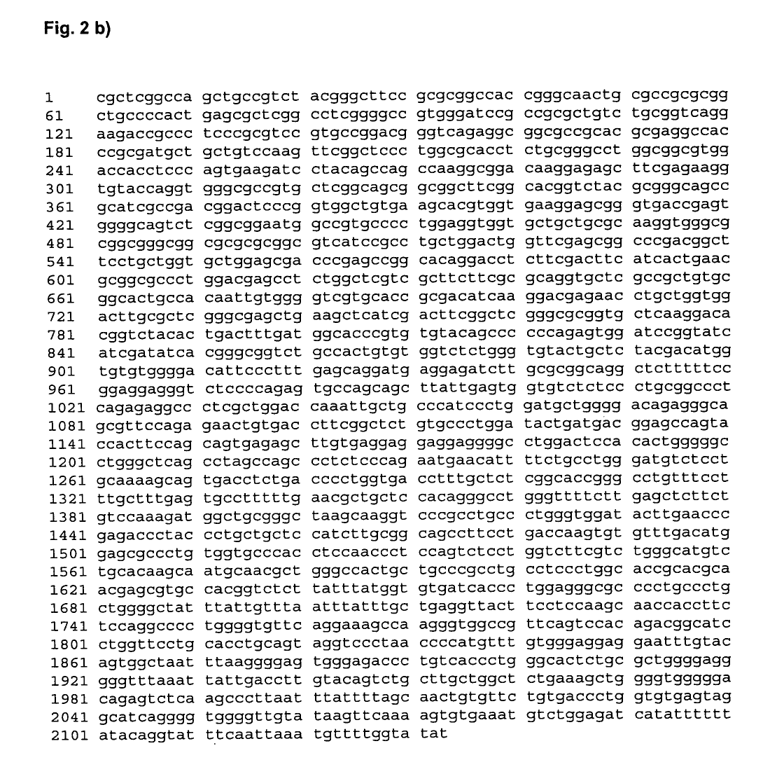

FIG. 2c) Amino acid sequence of PIM3-kinase, rat; AN: NM_{—}072124; SEQ ID NO. 9;

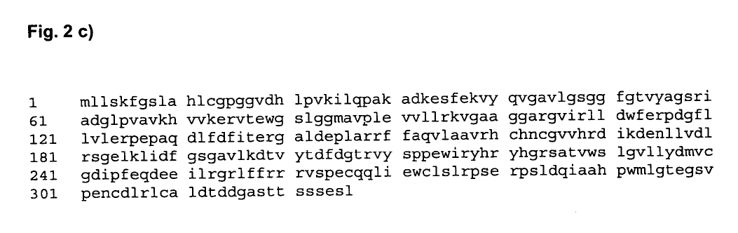

FIG. 2d) cDNA sequence of PIM3-kinase, mouse; AN: BC017621; SEQ ID NO. 10;

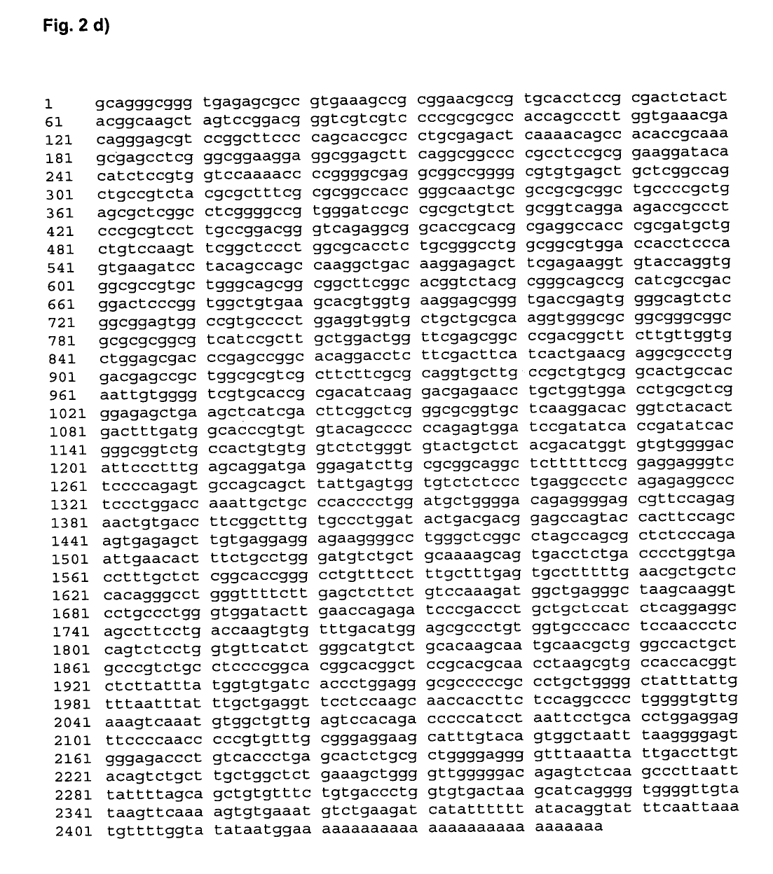

FIG. 2e) Amino acid sequence of PIM3-kinase, mouse; AN: BC017621; SEQ ID NO. 11;

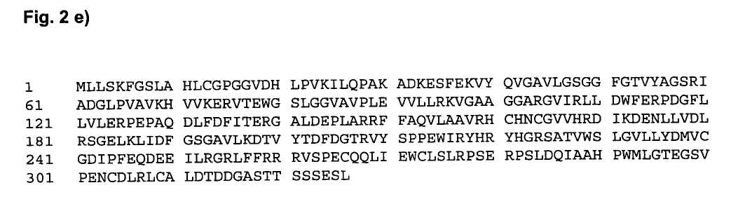

FIG. 3) mRNA expression of PIM-kinases in the lumbar marrow of the adult rat, see example 1);

FIG. 4) Changes in PIM1 gene expression in the spinal cord (L5) after ischiadicus ligature (Bennett); see example 1b);

FIG. 5) PIM1 mRNA level in the lumbar marrow (L5) after Bennett ligature, quantitative evaluation of the in situ hybridization results;

FIG. 6) mRNA expression pattern of PIM-kinases in the posterior horn of the spinal cord;

FIG. 7) mRNA expression of PIM-kinases in the spinal ganglion;

FIG. 8) Location of PIM1 gene expression in the spinal ganglion (L6) of the rat with in situ hybridization;

FIG. 9) Changes in the PIM1, PIM2 and PIM3 mRNA level in the spinal ganglion L6 after bilateral CFA arthritis; Densitometric analysis of the bands, stained with ethidium bromide, for PIM1, PIM2, PIM3 and GAPDH after electrophoretic separation of the PCR products (25 cycles); Measurement values shown as the quotient of PIM1 and GAPDH or PIM2/GAPDH or PIM3/GAPDH.

### EXAMPLES

### Example 1

### Identification of Pain-Regulated Genes

The following procedure was chosen:

The so-called formalin model in the rat in which formalin is injected into the rat paw was chosen as the starting point for isolation of pain-regulated genes. The target tissue in which the pain-regulated expression of the genes according to the invention was detected was the dorsal part of the spinal cord of the rat in segments L3-L6.

Animal model: The formalin test is a suitable model for the field of inflammatory/persistent pain (Duibisson et al., 1997). In this test, 50 μl 5% formalin solution were injected unilaterally into the hind paw of adult Wistar rats and the animals were sacrificed 24 hours after the injection for removal of tissue. In parallel, isotonic saline solution was injected into the hind paw in the control animals.

Tissue removal. The animals were decapitated, the spinal column removed, and sections of the spinal cord were prepared and hybridized with specific labeled antibodies against the PIM-kinases.

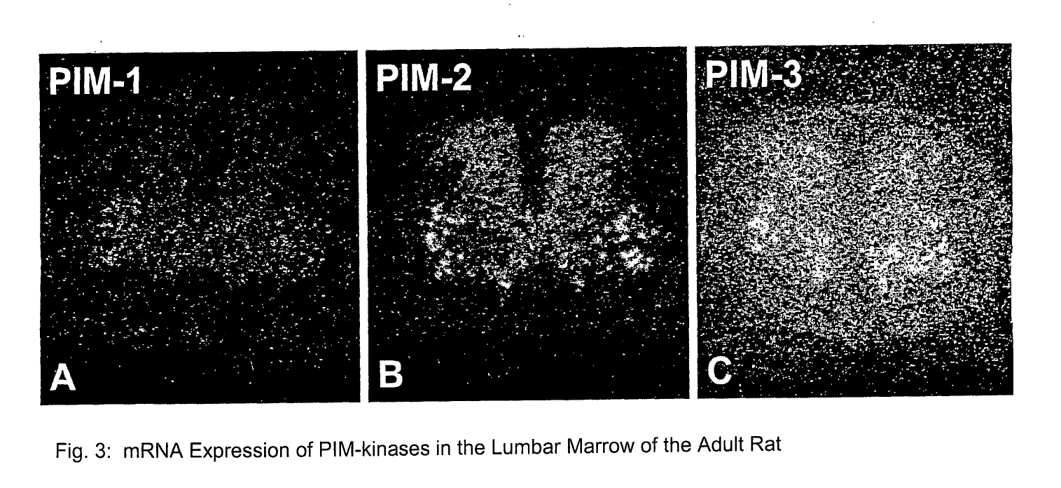

Example 1a) See FIG. 3

Digitalized X-ray film autoradiograms of frozen sections through the spinal cord (level L5) after in situ hybridization with specific ³⁵S-labeled RNA probes. The exposure time of the X-ray films was 72 hours. PIM1 is expressed to a relatively low degree under normal conditions (A). In contrast to this, PIM2 is expressed constitutively to a high degree (B) with a clear dominance in the grey matter (above all in the superficial posterior horn and in the motoneurons of the anterior horn). PIM3 mRNA is distributed over the entire section (C), with more intense signals over the neuronal regions and weak signals over the white matter.

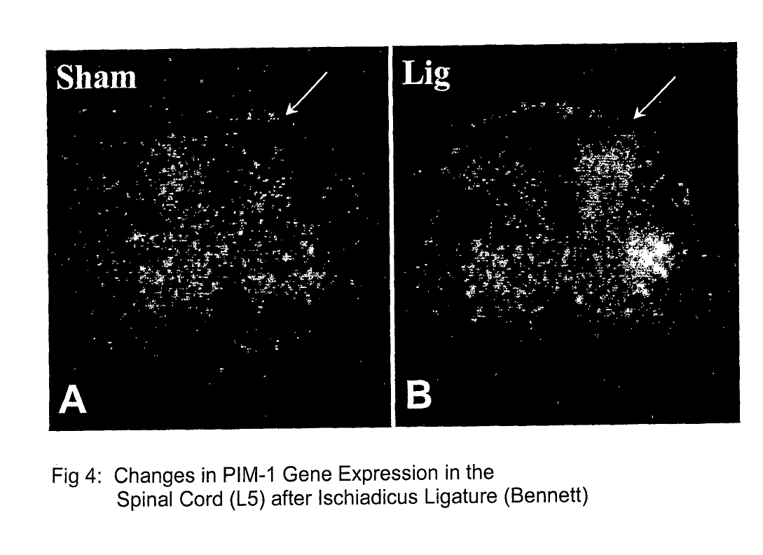

Example 1b) See FIG. 4

Increased PIM-1 gene expression in the spinal cord after ischiadicus ligature. Digitalized frozen sections through the lumbar marrow of animals 7 days after Bennett ligature (B) show, after hybridization with a radioactively labeled PIM1 probe, a clear increase in the PIM1 mRNA level in the spinal cord half ipsilaterally to the ligation (arrow), both in the posterior horn and in the anterior horn. In sham-operated animals (A), this increase is not observed.

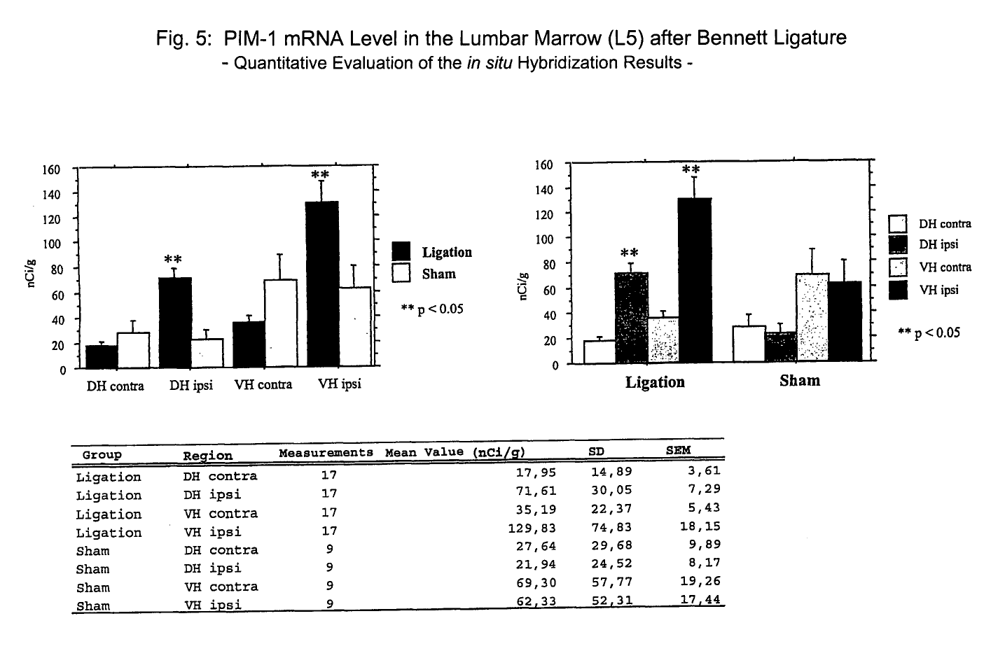

Example 1c) See FIG. 5

Semi-quantitative analysis of the PIM1 mRNA level in the various spinal cord regions of sham-operated animals and of animals 7 days after Bennett ligature.

After digitalization of the frozen sections hybridized with a PIM1-specific probe (MCID image analysis system, Imaging Research, Canada), the radioactive hybridization signals are recorded densitometrically. The measurement values are converted into nCi/g of tissue after a standard curve has been established.

To establish a standard curve, slides with ¹⁴C plastic strips with a defined radioactive content (American Radiolabeled Chemical Inc.) are exposed on X-ray film for the same duration together with the hybridized sections.

The contra- and ipsilateral regions of the posterior horn and of the anterior horn were in each case analyzed.

Nine measurements per region were carried out for the group of Sham-operated animals and 17 measurements for the group after Bennett ligature.

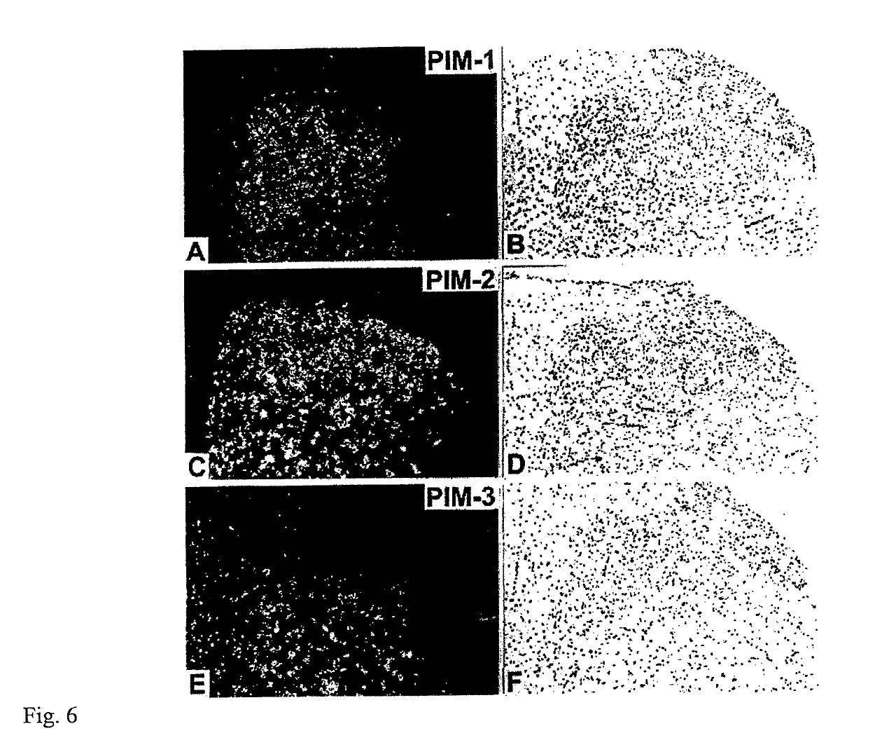

Example 1d) see FIG. 6

High-resolution dark-field photographs of the superficial posterior horn ipsilaterally to the ischiadicus ligature after hybridization with PIM-specific probes (A, C, E) and counter-staining with cresyl violet (B, D, F). PIM1-hybridized sections show a “layer”-specific expression of PIM1 in lamina 2 and 3 (A, B). PIM2 transcripts are expressed to a relatively high degree particularly in lamina 1 and lamina 2 and in the entire posterior horn (D, E). On the other hand, PIM3 is expressed less in the superficial posterior horn, but in lamina 3 and the deeper layers (E, F).

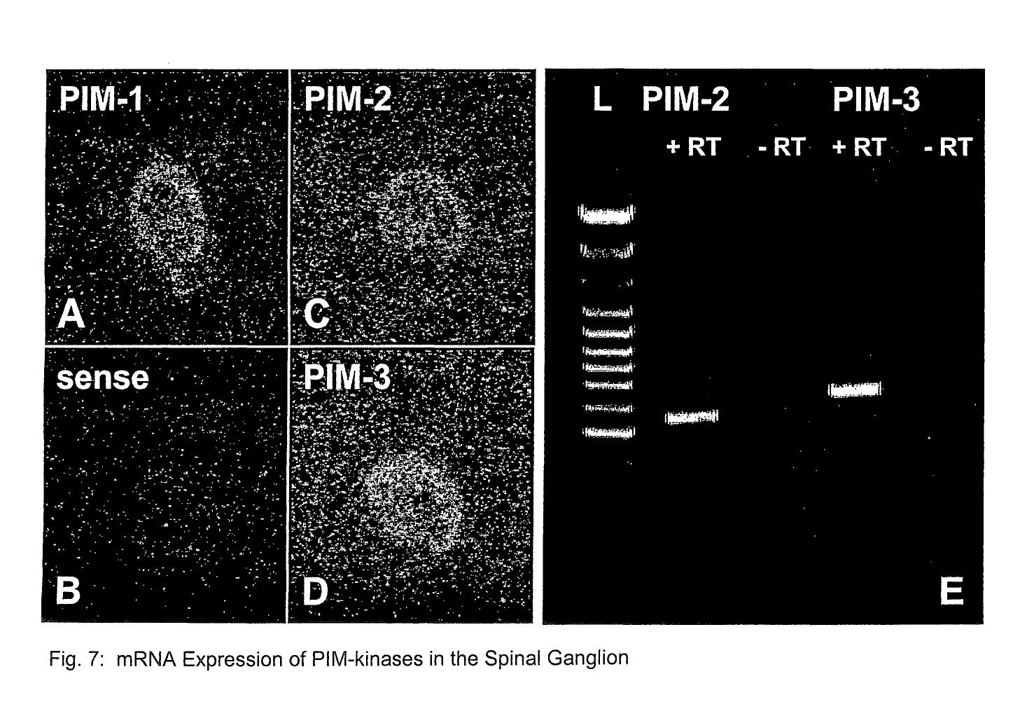

Example 1e) see FIG. 7

Digitalized X-ray film autoradiograms of frozen sections through a lumbar spinal ganglion (L6) after in situ hybridization with specific ³⁵S-labeled RNA probes. The exposure time of the X-ray films was 72 hours.

All three PIM-kinases are expressed in the spinal ganglion under normal conditions (A,C,D). Probes in sense orientation, shown here on the example for PIM1 (B), produce no specific hybridization signals. In comparison, PIM3 is expressed to a greater degree (D).

For the RT-PCR analysis, the spinal ganglia (L6) from 5 control animals were removed on both sides and pooled for extraction of the total RNA. Trizol (Gibco-BRL) was used for the RNA extraction. A Dnase I treatment was carried out before the reverse transcription was carried out.

For the reverse transcription with Superscript II (Gibco-BRL), 2.5 μg of the total RNA isolated were employed in a 50 μl reaction.

The PCR amplification was carried out in a Thermocycler 9700 (Perkin Elmer) in accordance with the following program:

1 cycle 95° C., 3 min; 40 cycles (94° C., 45 sec; 60° C., 45 sec; 72° C., 60 sec); 1 cycle 72° C., 7 min.

In each case 7.5 μl of the cDNA were employed as the template (50 ng/μl).

The specific amplicons for PIM2 (518 bp) and PIM3 (612 bp) had the expected sizes; GAPDH. RT reactions with omission of the reverse transcriptase were carried out as negative controls.

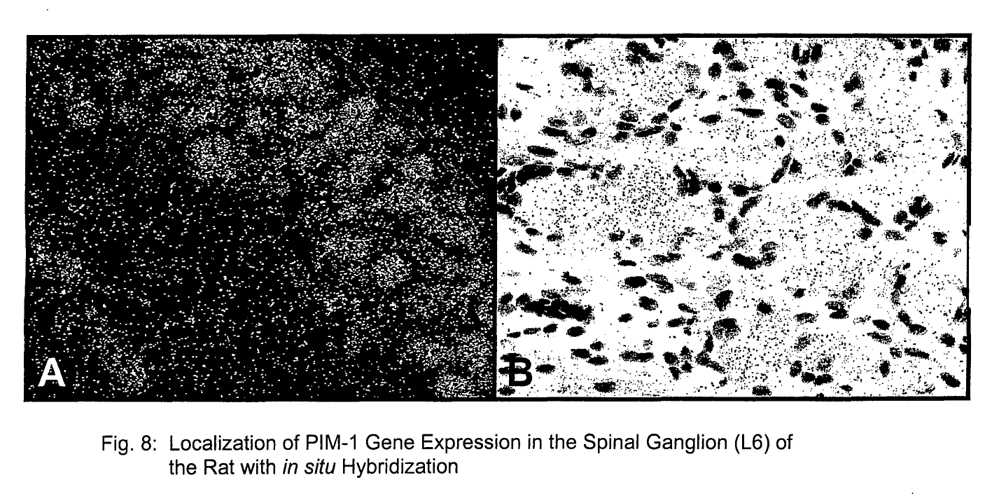

Example 1f) see FIG. 8

Hybridization of frozen sections with PIM1-specific probes shows specific hybridization signals over the cell-rich regions of the spinal ganglion (dark-field photograph in A). The microscopic high resolution in the light field confirms that the signals in the sections counter-stained with cresyl violet are primarily located over the neuronal cells (B).

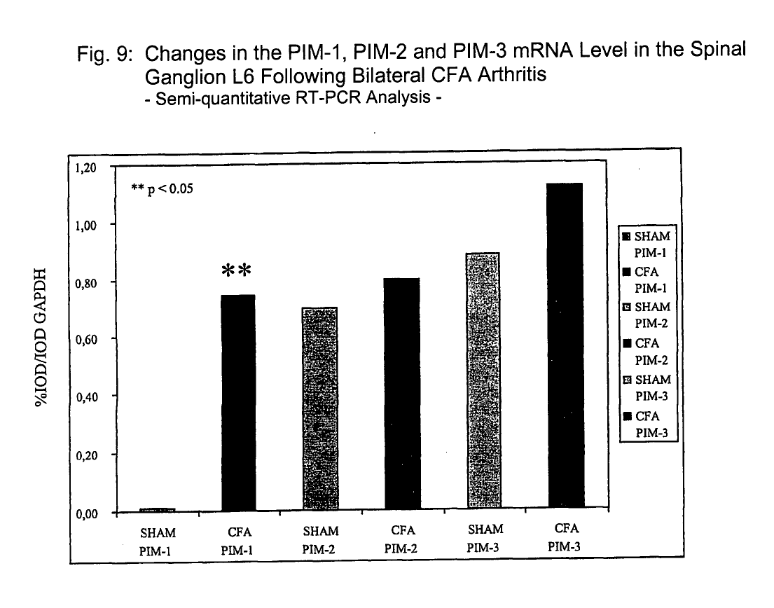

Example 1g) see FIG. 9

The spinal ganglia (L6) were removed from both sides from 5 animals and pooled for extraction of the total RNA.

Trizol (Gibco-BRL) was used for the RNA extraction. A Dnase I treatment was carried out before the reverse transcription was carried out.

For the reverse transcription with Superscript II (Gibco-BRL), 2.5 μg of the total RNA isolated were employed in a 50 μl reaction.

The PCR amplification was carried out in a Thermocycler 9700 (Perkin Elmer) in accordance with the following program:

1 cycle 95° C., 3 min; 40 cycles (94° C., 45 sec; 60° C., 45 sec; 72° C., 60 sec); 1 cycle 72° C., 7 min.

In each case 7.5 μl of the cDNA were employed as the template (50 ng/μl).

After 10, 15, 20, 25, 30, 35 and 40 cycles, in each case 10 μl of the PCR reaction were removed and separated by gel electrophoresis in order to determine the linear range of the amplification. The gels stained with ethidium bromide were digitalized and the PCR bands were measured densitometrically.

The specific amplicons had the expected sizes (PIM1, 550 bp; PIM2, 518 bp; PIM3, 612 bp; GAPDH, 227 bp).

To record the change in the PIM expression after CFA, the quotients of the specific amplicons (PIM1, PIM2, PIM3) and the non-regulated GAPDH were obtained for each test group.

Comprehensive Expression Analysis of All Three Members of the PIM-Kinase Family: PIM1, PIM2 and PIM3

The comprehensive expression analysis of all three members of the PIM-kinase family: PIM1, PIM2 and PIM3; is shown in tabular form below.

| Expres- | PIM1 | | PIM2 | | PIM3 | |
|---|---|---|---|---|---|---|
| sion | Neurons | Glia cells | Neurons | Glia cells | Neurons | Glia cells |
| Spinal | ++ | + | +++ | + | + | +++ |
| cord | | | | | | |
| DRG | ++ | + | +++ | + | + | +++ |

The ISH results show the following expression pattern for the PIM-kinases:

A neuronal location of PIM1, PIM2 and PIM3 mRNA in the spinal cord (ISH, RT-PCR)

A neuronal expression of PIM1, PIM2 and PIM3 mRNA in the DRG of the rat. PIM2 and PIM3 also appear to occur in non-neuronal cells. The immunohistochemical data show the PIM1 protein in DRG neurons and the PIM2 protein in DRG neurons and in glia cells.

For PIM1, an increased mRNA expression was detectable in several pain models:

Regulation upwards of the PIM1 mRNA in DRG extracts in the CFA model

Regulation upwards of PIM1 mRNA in the dorsal horn after ischiadicus ligature of the rat. In addition, an upwards regulation is also found in motor neuron areas of the anterior horn.

PIM1 in neuropathic pain regulation upwards in microglia (C1q) and in neurons

In contrast to PIM1, PIM2 is already expressed constitutively to quite a high degree in the posterior horn, the low regulation upwards observed in the Bennett model not being significant according to statistical evaluation.

Increase in the neuronal PIM1immune reactivity in the posterior horn in the Chung model.

The PIM3 mRNA is expressed in the deeper posterior horn and also in the anterior horn both neuronally and glially, after lesion but not regulated.

### Example 2

### Procedure for the Screening Method with Measurement of the Binding Via the Displacement of a Radioactively Labeled Ligand

A nucleic acid section which codes for PIM1-kinase is cloned in an expression vector which allows a constitutive expression (e.g. CMV promoter) or an inducible expression in eukaryotic cells. The DNA is introduced with suitable transfection processes, e.g. with Lipofectamin (Roche Diagnostics), into eukaryotic cells (e.g. CHO cells, HEK293 cells or NIH-3T3 cells). The cells are cultured in the presence of a selection reagent (e.g. zeocin, hygromycin or neomycin) such that only the cells which have taken up the DNA construct and, during longer-lasting selection, also incorporated it into the genome survive.

Starting from these cells, membrane fractions which contain PIM1-kinase in a large amount and can be used for a binding assay are obtained. This assay consists of 1.) the membranes containing PIM1-kinase, 2.) a radioactively labeled ligand, 3.) a binding buffer (e.g. 50 mM HEPES pH 7.4, 1 mM EDTA) and the ligand to be investigated for binding. After incubation of the abovementioned reaction mixtures (for e.g. 30-60 min) at a suitable temperature (usually room temperature), the non-bound radioactive ligand molecules are filtered off. The remaining amount of bound ligand is measured, after addition of a scintillation cocktail, in a β-counter (e.g. Trilux, Wallac). If the test substance shows binding to the PIM1-kinase, this is detected as a reduced radioactive incorporation. This method is suitably miniaturized such that it can be carried out in (96-, 384- or 1,536-well) microtiter plates in order to carry out this method by means of a robot in the so-called high throughput screening (HTS) method.

### Example 3

### Procedure for the Screening Method According to the Invention with Measurement of the Functional Parameters Modified by Binding of the Substance

A nucleic acid section which codes for the protein kinase PIM1 is cloned in an expression vector which allows an inducible expression in prokaryotes, such as e.g. E. coli. The nucleic acid section is modified here such that it is expressed as a fusion protein with an additional N- or C-terminal amino acid sequence. This sequence should allow, with a non-modified function of the PIM1 kinase, a purification via a specific method, e.g. glutathione S-transferase fragment, which allows isolation from the protein mixture via binding to glutathione. After transfection of the bacteria, induction of the gene (e.g. with IPTG in the case of the lac promoter) and breaking down of the bacteria, the fusion proteins are purified and employed in an in vitro kinase experiment. In this procedure, 5 μg protein are incubated at 30° C. for 30 minutes in 50 μl kinase buffer (20 mM PIPES, pH 7.0, 5 mM MnCl₂, 7 mM β-mercaptoethanol, 0.4 mM spermine, 10 mM rATP) supplemented with 10 μCi [γ³²P] ATP. Purified histone H1 protein (Sigma) or bacterially expressed GST-NFATc1 fusion protein are added as substrates. After the incubation time, the non-incorporated [γ-³²P] ATP is filtered off and the amount of ³²phosphate incorporated is determined by β-scintillation (Trilux, Wallac). In an experiment for discovering new PIM1-kinase inhibitors, the test substances are co-incubated in this batch and a decrease in the ³²P incorporation is used as an indicator for an inhibitor. This method is suitably miniaturized such that it can be carried out in (96-, 384- or 1,536-well) microtiter plates in order to carry out this method by means of a robot in the so-called high throughput screening (HTS) method.

### Example 4

### Example of a Medicament Comprising a Compound According to the Invention—Tablet Formulation

Tablets can be prepared by direct pressing of mixtures of the compound according to the invention with corresponding auxiliary substances or by pressing granules containing the compound (with optionally further auxiliary substances). The granules can be prepared here either by moist granulation with e.g. aqueous granulating liquids and subsequent drying of these granules or by dry granulation, e.g. via compacting

Direct pressing

| | | |
|---|---|---|
| e.g. per tablet: | 25 mg | compound according to the invention |
| | 271 mg | LUDIPRESS ™ (granules for direct tablet |
| | | making from lactose monohydrate, |
| | | povidone K30 and crospovidone) |
| | 4 mg | magnesium stearate |
| | 300 mg | total |

Prepare a homogeneous mixture of the active compound with the auxiliary substances and press this on a tablet press to give tablets with a diameter (Ø) of 10 mm.

Dry granulation

| | | |
|---|---|---|
| e.g. per tablet: | 25 mg | compound according to the invention |
| | 166 mg | microcrystalline cellulose |
| | 80 mg | hydroxypropylcellulose with a low degree of |
| | | substitution (I-HPC LH 11 ™) |
| | 5 mg | highly disperse silicon dioxide |
| | 4 mg | magnesium stearate |
| | 280 mg | total |

Prepare a homogeneous mixture of the compound with the microcrystalline cellulose and the I-HPC and compact this. After sieving of the compressed bodies, the granules formed are mixed with magnesium stearate and silicon dioxide and pressed on a tablet press to give tablets with a Ø of 9 mm.

Moist granulation

| | | |
|---|---|---|
| e.g. per tablet: | 25 mg | compound according to the invention |
| | 205 mg | microcrystalline cellulose |
| | 6 mg | povidone K30 |
| | 10 mg | crospovidone |
| | 4 mg | magnesium stearate |
| | 250 mg | total |

Prepare a homogeneous mixture of the compound with the microcrystalline cellulose and the crospovidone and granulate this with an aqueous solution of the povidone in a granulator. The moist granules are then after-granulated and, after drying, dried in a drying cabinet (50° C.) for 10 h. The dry granules are sieved together with the magnesium stearate, finally mixed and pressed on a tablet press to give tablets with a Ø of 8 mm.

### Example 5

### Example of a Medicament Comprising a Compound According to the Invention—Parenteral Solution

1 g of a compound according to the invention is dissolved in 1 l water for injection purposes at room temperature and the solution is then adjusted to isotonic conditions by addition of NaCl (sodium chloride).

The foregoing description and examples have been set forth merely to illustrate certain embodiments of the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

### Literature

The following documents are incorporated herein by reference in their entirety.

Akopian A N, Sivilotti L & Wood J N (1995) Nature 379:257-262.

Ausubel F M, Brent R, Kingdton R E, Moore D D, Seidman J G, Smith J A & Struhl K eds. (1990) Current protocols in molecular biology. John Wiley & Sons, Inc. New York, N.Y.

Baba H, Doubell T P, Woolf C J 1999: Peripheral inflammation facilitates Aβ fiber-mediated synaptic input to the substantia gelatinosa of the adult rat spinal cord. J Neurosci 19:859-867.

Bauer D, Müller H, Reich J, Riedel H, Ahrenkiel V, Warthoe P & Strauss M (1993): Identification of differentially expressed mRNA species by an improved display technique (DDRT-PCR) Nucl Acids Res 21:4272-4280.

Bonini A, Anderson S M, Steiner D F (1997) Molecular cloning and tissue expression of a novel orphan G protein-coupled receptor from rat lung. Biochem Biophys Res Comm 234:190-193.

Chih-Cheng et al., (1995): A P2X prinoceptor expressed by a subset of sensory neurons. Nature 377:428-432.

Corderre T J, Katz J, Vaccarino A L, Melzack R (1993): Contribution of central plasticity to pathological pain: review of clinical and experimental evidence. Pain 52:259-285.

Dickenson (1995) Novel pharmacological targets in the treatment of pain. Pain Rev., 2, 1-12.

Dubuisson et al., 1997 Pain, 4:161-174.

Feng Y & Gregor P (1997) Cloning of a novel member of the G protein-coupled receptor family related to peptide receptors. Biochem Biophys Res Comm 231:651-654.

Furukawa T, Yang Y, Nakamoto B, Stamatoyannopoulos G, Papayannopoulou T (1966): Identification of new genes expressed in a human erythroleukemia cell line. Bloods Cell Mol & Dis 22:11-22.

Gunasekar P G, Kanthasamy, A G, Borowitz J L, Isom G E 1995: NMDA receptor activation produces concurrent generation of nitric oxide and reactive oxygen species: implication for cell death. J Neurochem 65:2016-2021.

Hawes B E, Fried S, Yao X, Weig B, Graziano M P 1998: Nociceptin (ORL1) and μ-opioid receptors mediate mitogen-activated protein kinase activation in CHO cells through a Gi-coupled signaling pathway: evidence for distinct mechanisms of agonist-mediated desensitization. J Neurochem 71:1024-1033.

Hubank M & Schatz D G (1994): Identifying differences in mRNA expression by representational difference analysis of cDNA. Nucl Acids Res 22:5640-5648.

Kluβmann S et al., 1996: Nature Biotechnology 14:1112-1115.

Li L-Y & Chang K-J 1996: The stimulatory effect of opioids on mitogen-activated protein kinase in chinese hamster ovary cells transfected to express μ-opioid receptors. Mol Pharm 50:599-602.

Lian P & Pardee A B 1992: Differential display of eukaryotic messenger RNA by means of the polymerase chain reaction. Science 257:967-971.

Methner A, Hermey G, Schinke B, Hermanns-Borgmeyer I (1997): A novel G protein-coupled receptor with homology to neuropeptide and chemoattractant receptors expressed during bone development. Biochem Biophys Res Comm 233:336-342.

Mohit A A, Martin J H & Miller C A 1995: p493F12 kinase: a novel MAP kinase expressed in a subset of neurons in the human nervous system. Neuron 14:67-78.

Poirier G M-C, Pyati J, Wan J S, Erlander M G 1997: Screening differentially expressed cDNA clones obtained by differential display using amplified RNA. Nucleic Acids Research 25:913-914.

Sambrook J, Fritsch E F & Maniatis T 1989: Molecular Cloning: A Laboratory Manual. Second Edition. Cold Spring Harbor Laboratory Press, Cold Spring Harbor.

Sompayrac L, Jane S, Burn T C, Tenen D G & Danna K J 1995: Overcoming limitations of the mRNA differential display technique. Nucleic Acids Research 23:4738-4739.

Tal M 1996: A novel antioxidant alleviates heat hyperalgesia in rats with an experimental painful neuropathy. Neurreport 7:1382-1384.

Tölle T R (1997): Chronischer Schmerz. In: Klinische Neurobiologie [Chronic Pain. In: Clinical Neurobiology], Herdergen T. Tölle T R, Bähr M (eds.): p. 307-336; Spektrum Verlag, Heidelberg.

U.S. Pat. No. 5,262,311, issued Nov. 16, 1993.

Velculescu V E, Zhang L, Vogelstein B, Kinzler K W (1995): Serial analysis of gene expression. Science 270:484-487.

Wan J S, Sharp J S et al. (1996): Cloning differentially expressed mRNAs. Nature Biotech 14:1685-1691.

Watson J B & Margulies J E (1993) Differential cDNA screening strategies to identify novel stage-specific proteins in the developing mammalian brain. Dev Neurosci 15:77-86.

Wilks A F (1989) Two putative protein-tyrosine kinases identified by application of the polymerase chain reaction. Proc Natl Acad Sci USA 86:1603-1607.

Woolf C J, Shortland P, Coggeshall R E 1992: Peripheral nerve injury triggers central sprouting of myelinated afferents. Nature 355:75-78.

Zimmermann, M & Herdegen, T (1996): Plasticity of the nervous system at the systemic, cellular and molecular levels: a mechanism of chronic pain and hyperalgesia. Progr Brain Res 110:233-259.

What is claimed is:

1. A method for detecting a pain-regulating substance comprising the steps of:
(a) incubating a test substance with a cell or a preparation from a cell which has synthesized the protein PIM1-kinase or PIM3-kinase or a protein comprising SEQ ID NO: 2, 4, 6, 9 or 11 or a protein which is at least 90% homologous thereto or a protein encoded by a polynucleotide comprising SEQ ID NO: 1, 3, 5, 7, 8 or 10 or a protein encoded by a polynucleotide which is at least 90% homologous thereto, or a protein encoded by a nucleic acid which binds under stringent conditions to a polynucleotide comprising SEQ ID NO: 1, 3, 5, 7, 8 or 10 or antisense polynucleotides thereof, or a part protein of one of the abovementioned proteins which is at least 10 amino acids long and
(b) measuring the binding of the test substance to the protein or part protein synthesized by the cell or measuring at least one functional parameter modified by the binding of the test substance to the protein or part protein.

2. A method according to claim 1, wherein the cell is manipulated by genetic engineering before step (a).

3. A method according to claim 2, wherein the manipulation by genetic engineering allows the measurement of at least one functional parameter modified by the binding of the test substance.

4. A method according to claim 3, wherein the manipulation by genetic engineering causes expression of a form of a G protein which is not expressed endogenously in the cell or introduction of a reporter gene.

5. A method according to claim 2, wherein the cell is manipulated by genetic engineering so that the cell contains at least one polynucleotide selected from the group consisting of SEQ ID NOS: 1, 3, 5, 7, 8 and 10 or a polynucleotide which is at least 90% homologous thereto.

6. A method according to claim 5, wherein the polynucleotide is contained in a recombinant DNA construct.

7. A method according to claim 2, wherein after the manipulation by genetic engineering and before step (a), the cell is cultured under conditions which allow expression.

8. A method according to claim 7, wherein the cell is cultured under selection pressure.

9. A method according to claim 1, wherein the cell is an amphibia cell, bacteria cell, yeast cell, insect cell or an immortalized or native mammalian cell.

10. A method according to claim 1, wherein the measuring of the binding is carried out via the displacement of a known labeled ligand of the part protein or protein or via the activity bound thereto from a labeled test substance.

11. A method according to claim 1, wherein the measuring of at least one functional parameter modified by the binding of the test substance is carried out via measurement of the regulation, inhibition or activation of receptors, ion channels or enzymes.

12. A method according to claim 1, wherein the measuring of at least one functional parameter modified by the binding of the test substance is carried out via measurement of the modification of the gene expression, the ionic medium, the pH, the membrane potential, the enzyme activity or the concentration of the second messenger.

13. A method according to claim 1, wherein the protein or part protein in steps (a) and (b) is:
PIM1-kinase;
a protein encoded by a polynucleotide comprising SEQ ID NO: 1, 3 or 5 or a protein encoded by a polynucleotide which is at least 90% homologous thereto;
a protein with an amino acid sequence comprising SEQ ID NO: 2, 4 or 6 or a protein which is at least 90% homologous thereto;
a protein which is coded by a nucleic acid which binds under stringent conditions to a polynucleotide comprising SEQ ID NO: 1, 3 or 5 or antisense polynucleotides thereof, or
a part protein of one of the abovementioned proteins which is at least 10 amino acids long.

14. A method according to claim 13, wherein the protein or part protein in steps (a) and (b) is:
a protein encoded by a polynucleotide which is at least 95% homologous to a polynucleotide comprising SEQ ID NO: 1, 3 or 5;
a protein with an amino acid sequence which is at least 95% homologous to a protein comprising SEQ ID NO: 2, 4 or 6, or
a part protein of one of the abovementioned proteins which is at least 10 amino acids long.

15. A method according to claim 14, wherein the protein or part protein in steps (a) and (b) is:
a protein encoded by a polynucleotide which is at least 97% homologous to a polynucleotide comprising SEQ ID NO: 1, 3 or 5;
a protein with an amino acid sequence which is at least 97% homologous to a protein comprising SEQ ID NO: 2, 4 or 6, or
a part protein of one of the abovementioned proteins which is at least 10 amino acids long.

16. A polynucleotide which is at least 90% homologous with SEQ ID NO: 7 or SEQ ID NO: 10.

17. A polynucleotide according to claim 16, which is at least 95% homologous with SEQ ID NO: 7 or SEQ ID NO: 10.

18. A polynucleotide according to claim 17, which is at least 97% homologous with SEQ ID NO: 7 or SEQ ID NO: 10.

19. An antisense polynucleotide or peptidic nucleic acid which is capable of binding specifically to a polynucleotide according to claim 16.

20. A vector comprising a polynucleotide which is at least 90% homologous with SEQ ID NO: 7 or SEQ ID NO: 10 or a polynucleotide which is an antisense polynucleotide or peptidic nucleic acid which is capable of binding specifically to a polynucleotide which is at least 90% homologous with SEQ ID NO: 7 or SEQ ID NO: 10.

21. A protein encoded by a polynucleotide according to claim 16.

22. A protein encoded by a polynucleotide which hybridizes under stringent conditions with SEQ ID NO: 7 or SEQ ID NO: 10 or an antisense polynucleotide thereof.

23. A protein which is at least 90% homologous with SEQ ID NO: 11.

24. The protein of claim 23, wherein said protein is at least 95% homologous with SEQ ID NO: 11.

25. The protein of claim 24, wherein said protein is at least 97% homologous with SEQ ID NO: 11.

26. An antibody against a protein wherein said protein is:
encoded by a polynucleotide which is at least 90% homologous with SEQ ID NO: 7 or 10;
encoded by a polynucleotide which hybridizes under stringent conditions with SEQ ID NO: 7 or 10 or an antisense polynucleotide thereof or
at least 90% homologous with SEQ ID NO: 11.

27. A cell comprising:
a polynucleotide which is at least 90% homologous with SEQ ID NO: 7 or SEQ ID NO:10;
an antisense polynucleotide or peptidic nucleic acid capable of binding specifically to a polynucleotide which is at least 90% homologous with SEQ ID NO: 7 or SEQ ID NO: 10;
a protein encoded by a polynucleotide which is at least 90% homologous with SEQ ID NO: 7 or SEQ ID NO: 10;
a protein encoded by a polynucleotide which hybridizes under stringent conditions with SEQ ID NO: 7 or SEQ ID NO: 10 or a protein encoded by an antisense polynucleotide thereof;
a protein which is at least 90% homologous with SEQ ID NO: 11, or
a vector comprising a polynucleotide which is at least 90% homologous with SEQ ID NO: 7 or SEQ ID NO: 10 or an antisense polynucleotide or peptidic nucleic acid which is capable of binding specifically to a polynucleotide which is at least 90% homologous with SEQ ID NO: 7 or SEQ ID NO: 10.

28. A transgenic non-human mammal, the germ and somatic cells of which comprise:
a nucleotide sequence which is at least 90% homologous with SEQ ID NO: 7 or SEQ ID NO: 10, or
an antisense polynucleotide or peptidic nucleic acid capable of binding specifically to a polynucleotide which is at least 90% homologous with SEQ ID NO: 7 or SEQ ID NO: 10.

29. A transgenic non-human mammal according to claim 28, wherein said mammal is a rodent.

30. A transgenic non-human mammal, the germ and somatic cells of which no longer contain one of the nucleotide sequences according to claim 16 in an expressible form as a result of a chromosomal manipulation in the genome of said mammal or in the genome of an ancestor of said mammal.

31. A transgenic non-human mammal according to claim 30, wherein said mammal is a rodent.

32. The method of claim 1, further comprising the step of comparing the measurements obtained after repeating steps (a) and (b) wherein in at least part of the method the protein or part protein in steps (a) and (b) is:
PIM1-kinase;
a protein encoded by a polynucleotide comprising SEQ ID NO: 1, 3 or 5 or a polynucleotide which is at least 90% homologous thereto;
a protein with an amino acid sequence comprising SEQ ID NO: 2, 4 or 6, or a protein which is at least 90% homologous thereto;
a protein encoded by a nucleic acid which binds under stringent conditions to a polynucleotide comprising SEQ ID NO: 1, 3 or 5 or antisense polynucleotides thereof, or
a part protein of one of the abovementioned proteins which is at least 10 amino acids long,
or in another part of the method the protein or part protein in steps (a) and (b) is:
PIM2-kinase;
PIM3-kinase;
a protein encoded by a polynucleotide comprising SEQ ID NO: 7, 8 or 10 or a polynucleotide at least 90% homologous thereto;
a protein with an amino acid sequence comprising SEQ ID NO: 9 or 11 or a protein which is at least 90% homologous thereto;
a protein encoded by a nucleic acid which binds under stringent conditions to a polynucleotide comprising SEQ ID NO: 7, 8 or 10 or antisense polynucleotides thereof, or
a part protein of one of the abovementioned proteins which is at least 10 amino acids long.

33. The method of claim 32, wherein in at least part of the method the protein or part protein in steps (a) and (b) is:
a protein encoded by a polynucleotide which is at least 95% homologous to SEQ ID NO: 1, 3 or 5;
a protein which is at least 95% homologous to SEQ ID NO: 2, 4 or 6, or
a part protein of one of the abovementioned proteins which is at least 10 amino acids long,
or in another part of the method the protein or part protein in steps (a) and (b) is:
a protein encoded by a polynucleotide which is at least 95% homologous to SEQ ID NO: 7, 8 or 10;
a protein which is at least 95% homologous to SEQ ID NO: 9 or 11, or
a part protein of one of the abovementioned proteins which is at least 10 amino acids long.

34. The method of claim 32, wherein in at least part of the method the protein or part protein in steps (a) and (b) is:
a protein encoded by a polynucleotide which is at least 97% homologous to SEQ ID NO: 1, 3 or 5;
a protein which is at least 97% homologous to SEQ ID NO: 2, 4 or 6, or
a part protein of one of the abovementioned proteins which is at least 10 amino acids long,
or in another part of the method the protein or part protein in steps (a) and (b) is:
a protein encoded by a polynucleotide which is at least 97% homologous to SEQ ID NO: 7, 8 or 10;
a protein which is at least 97% homologous to SEQ ID NO: 9 or 11, or
a part protein of one of the abovementioned proteins which is at least 10 amino acids long.

35. A compound identified as a pain-regulating substance by the steps of:
(a) incubating a test substance with a cell or a preparation from a cell which has synthesized the protein PIM1-kinase or PIM3-kinase or a protein comprising SEQ ID NO: 2, 4, 6, 9 or 11 or a protein which is at least 90% homologous thereto or a protein encoded by a polynucleotide comprising SEQ ID NO: 1, 3, 5, 7, 8 or 10 or a protein encoded by a polynucleotide which is at least 90% homologous thereto, or a protein encoded by a nucleic acid which binds under stringent conditions to a polynucleotide comprising SEQ ID NO: 1, 3, 5, 7, 8 or 10 or an antisense polynucleotide thereof, or a part protein of one of the abovementioned proteins which is at least 10 amino acids long, and
(b) measuring the binding of the test substance to the protein or part protein synthesized by the cell, or measuring at least one functional parameter modified by the binding of the test substance to the protein or part protein.

36. A composition comprising as a pharmaceutically or diagnostically active ingredient an effective amount of:
a. a polynucleotide which codes for PIM1-kinase or PIM3-kinase or a polynucleotide which is at least 90% homologous with SEQ ID NO. 1, 3, 5, 7, 8 or 10;
b. a polynucleotide which is capable of binding specifically to one of the polynucleotides listed under point a);
c. a vector containing a polynucleotide according to one of points a) or b);
d. a PIM1-kinase or PIM3-kinase or a protein comprising SEQ ID NO: 2, 4, 6, 9 or 11 or a protein which is at least 90% homologous with one of these abovementioned proteins or a protein encoded by a polynucleotide comprising SEQ ID NO: 1, 3, 5, 7, 8 or 10 or a protein encoded by a polynucleotide which is at least 90% homologous thereto, or a protein encoded by a nucleic acid which binds under stringent conditions to a polynucleotide comprising SEQ ID NO: 1, 3, 5, 7, 8 or 10 or a protein encoded by antisense polynucleotides thereof or a part protein of one of the abovementioned proteins which is at least 10 amino acids long;
e. an antibody against one of the proteins or part proteins according to point d);
f. a cell containing a polynucleotide according to one of points a) or b), a vector according to point c), a protein or part protein according to point d) or an antibody according to point e);
g. a compound according to claim 35, or
h. an active compound which binds to a protein or part protein according to point d), and
at least one carrier or auxiliary substance.

37. The composition of claim 36, wherein said composition comprises:
a. a polynucleotide which is at least 95% homologous with SEQ ID NO: 1, 3, 5, 7, 8 or 10;
b. a polynucleotide which is capable of binding specifically to one of the polynucleotides listed under point a);
c. a vector containing a polynucleotide according to point a) or b), or
d. a cell containing a polynucleotide according to point a) or b), or a vector according to point c).

38. The composition of claim 36, wherein said composition comprises:
a. a polynucleotide which is at least 97% homologous with SEQ ID NO: 1, 3, 5, 7, 8 or 10;
b. a polynucleotide which is capable of binding specifically to one of the polynucleotides listed under point a);
c. a vector containing a polynucleotide according to point a) or b), or
d. a cell containing a polynucleotide according to point a) or b), or a vector according to point c).

39. The composition of claim 36, wherein said composition comprises an antisense polynucleotide or a peptidic nucleic acid capable of binding specifically to one of the polynucleotides listed under point a).

40. A method of alleviating pain in a mammal, said method comprising administering to said mammal an effective pain alleviating amount of a composition according to claim 36.

41. The method of claim 40, wherein said pain is chronic pain.

42. The method of claim 40, wherein said pain is neuropathic pain or inflammation-induced pain.

43. A method of providing gene therapy to a mammal, said method comprising administering to said mammal a therapeutic amount of:
a. a polynucleotide which codes for PIM1-kinase or PIM3-kinase or a polynucleotide which is at least 90% homologous with SEQ ID NO: 1, 3, 5, 7, 8 or 10;
b. a polynucleotide which is capable of binding specifically to one of the polynucleotides listed under point a);
c. a vector comprising a polynucleotide according to point a) or b), or
f. a cell containing a polynucleotide according to point a) or b), or a vector according to point c).

44. The method of claim 43, wherein said gene therapy is in vivo gene therapy.

45. The method of claim 43, wherein said gene therapy is in vitro gene therapy.

46. A method of diagnosing a mammal, said method comprising administering to said mammal the active ingredient of claim 36, and measuring a change in a functional parameter caused by said active ingredient.

47. A method for investigating the activity of a test substance comprising the steps of:
incubating a test substance with the active ingredient of claim 36, and
measuring binding of the test substance with the active ingredient, or measuring at least one functional parameter modified by interaction of the test substance with the active ingredient.

48. The composition of claim 36 wherein
the polynucleotide according to point a) is a polynucleotide which is at least 90% homologous with SEQ ID NO: 1, 3 or 5 or
the protein according to point d) is a protein comprising SEQ ID NO: 2, 4 or 6, or a protein which is at least 90% homologous to SEQ ID NO: 2, 4 or 6, or a protein encoded by a polynucleotide comprising SEQ ID NO: 1, 3 or 5, or a protein encoded by a polynucleotide which is at least 90% homologous to SEQ ID NO: 1, 3 or 5, or a protein encoded by a nucleic acid which binds under stringent conditions to a polynucleotide comprising SEQ ID NO: 1, 3 or 5 or antisense polynucleotides thereof, or a part protein of one of the abovementioned proteins which is at least 10 amino acids long.

49. The method of alleviating pain of claim 40 wherein
the polynucleotide according to point a) is a polynucleotide which is at least 90% homologous with SEQ ID NO: 1, 3 or 5, or
the protein according to point d) is a protein comprising SEQ ID NO: 2, 4 or 6, or a protein which is at least 90% homologous to SEQ ID NO: 2, 4 or 6, or a protein encoded by a polynucleotide comprising SEQ ID NO: 1, 3 or 5, or a protein encoded by a polynucleotide which is at least 90% homologous to SEQ ID NO: 1, 3 or 5, or a protein encoded by a nucleic acid which binds under stringent conditions to a polynucleotide comprising SEQ ID NO: 1, 3 or 5 or antisense polynucleotides thereof, or a part protein of one of the abovementioned proteins which is at least 10 amino acids long.

50. The method of providing gene therapy of claim 43, wherein
the polynucleotide according to point a) is a polynucleotide which is at least 90% homologous with SEQ ID NO: 1, 3 or 5, or
the protein according to point d) is a protein comprising SEQ ID NO: 2, 4 or 6, or a protein which is at least 90% homologous to SEQ ID NO: 2, 4 or 6, or a protein encoded by a polynucleotide comprising SEQ ID NO: 1, 3 or 5, or a protein encoded by a polynucleotide which is at least 90% homologous to SEQ ID NO: 1, 3 or 5, or a protein encoded by a nucleic acid which binds under stringent conditions to a polynucleotide comprising SEQ ID NO: 1, 3 or 5 or antisense polynucleotides thereof, or a part protein of one of the abovementioned proteins which is at least 10 amino acids long.

51. The method of diagnosing a mammal of claim 46, wherein
the polynucleotide according to point a) is a polynucleotide which is at least 90% homologous with SEQ ID NO: 1, 3 or 5, or
the protein according to point d) is a protein comprising SEQ ID NO: 2, 4 or 6, or a protein which is at least 90% homologous to SEQ ID NO: 2, 4 or 6, or a protein encoded by a polynucleotide comprising SEQ ID NO: 1, 3 or 5, or a protein encoded by a polynucleotide which is at least 90% homologous to SEQ ID NO: 1, 3 or 5, or a protein encoded by a nucleic acid which binds under stringent conditions to a polynucleotide comprising SEQ ID NO: 1, 3 or 5 or antisense polynucleotides thereof, or a part protein of one of the abovementioned proteins which is at least 10 amino acids long.

52. The method of investigating activity according to claim 47, wherein
the polynucleotide according to point a) is a polynucleotide which is at least 90% homologous with SEQ ID NO: 1, 3 or 5, or
the protein according to point d) is a protein comprising SEQ ID NO: 2, 4 or 6, or a protein which is at least 90% homologous to SEQ ID NO: 2, 4 or 6, or a protein encoded by a polynucleotide comprising SEQ ID NO: 1, 3 or 5, or a protein encoded by a polynucleotide which is at least 90% homologous to SEQ ID NO: 1, 3 or 5, or a protein encoded by a nucleic acid which binds under stringent conditions to a polynucleotide comprising SEQ ID NO: 1, 3 or 5 or antisense polynucleotides thereof, or a part protein of one of the abovementioned proteins which is at least 10 amino acids long.

53. The polynucleotide of claim 16, wherein said polynucleotide is selected from the group consisting of RNA, single-stranded DNA and double-stranded DNA.

54. The polynucleotide of claim 53, wherein said polynucleotide is an mRNA or a cDNA.

55. The polynucleotide of claim 19, wherein said polynucleotide is selected from the group consisting of RNA, single-stranded DNA and double-stranded DNA.

56. The polynucleotide of claim 55, wherein said polynucleotide is an mRNA or a cDNA.

57. The polynucleotide of claim 19, wherein said polynucleotide is part of a DNA enzyme or a catalytic RNA or DNA.

58. The polynucleotide of claim 57, wherein said polynucleotide is part of a ribozyme.

59. The composition of claim 36, wherein said active ingredient is a polynucleotide selected from the group consisting of RNA, single-stranded DNA and double-stranded DNA.

60. The composition of claim 59, wherein said polynucleotide is an mRNA or a cDNA.

61. The composition of claim 36 wherein said active ingredient is a polynucleotide which is part of a DNA enzyme or a catalytic RNA or DNA.

62. The composition of claim 61, wherein said polynucleotide is part of a ribozyme.

63. The vector of claim 20, wherein said vector is an expression vector.

64. The vector of claim 20, wherein said vector is derived from a virus or said vector contains at least one LTR, poly A, promoter, or ORI sequence.

65. The composition of claim 36, wherein said vector is an expression vector.

66. The composition of claim 36, wherein said vector is derived from a virus, or said vector contains at least one LTR, poly A, promoter, or ORI sequence.

67. The protein of claim 21, wherein said protein has been post-translationally modified.

68. The protein of claim 67 wherein said protein has been glycosylated, phosphorylated, amidated, methylated, acetylated, ADP-ribosylated, hydroxylated, provided with a membrane anchor, cleaved or shortened.

69. The protein of claim 22, wherein said protein has been post-translationally modified.

70. The protein of claim 69, wherein said protein has been glycosylated, phosphorylated, amidated, methylated, acetylated, ADP-ribosylated, hydroxylated, provided with a membrane anchor, cleaved or shortened.

71. The protein of claim 23, wherein said protein has been post-translationally modified.

72. The protein of claim 71, wherein said protein has been glycosylated, phosphorylated, amidated, methylated, acetylated, ADP-ribosylated, hydroxylated, provided with a membrane anchor, cleaved or shortened.

73. The composition of claim 36, wherein said active ingredient is a protein or part protein which has been post-translationally modified.

74. The composition of claim 73, wherein said protein or part protein has been glycosylated, phosphorylated, amidated, methylated, acetylated, ADP-ribosylated, hydroxylated, provided with a membrane anchor, cleaved or shortened.

75. The antibody of claim 26, wherein said antibody is a monoclonal antibody.

76. The antibody of claim 26, wherein said antibody is a polyclonal antibody.

77. The composition of claim 36, wherein said active ingredient is a monoclonal antibody.

78. The composition of claim 36, wherein said active ingredient is a polyclonal antibody.

79. The cell of claim 27, wherein said cell is an amphibia cell, bacteria cell, yeast cell, insect cell or an immortalized or native mammalian cell.

80. The composition of claim 36 wherein said active ingredient is an amphibia cell, bacteria cell, yeast cell, insect cell or an immortalized or native mammalian cell.

81. The compound of claim 35, wherein said compound is a low molecular weight compound.

82. The composition of claim 36, wherein said active ingredient is a low molecular weight compound.
